(19) United States
(12) Patent Application Publication
(10) Pub. No.: US 2021/0298981 A1
(43) Pub. Date: Sep. 30, 2021

(54) SURGICAL BED, ENDOSCOPIC SURGICAL DEVICE, ENDOSCOPIC SURGICAL METHOD, AND SYSTEM

(71) Applicants: Medicaroid Corporation, Kobe-shi (JP); Ziosoft, Inc., Tokyo (JP)

(72) Inventors: Jota IDA, Kobe-shi (JP); Mitsuichi HIRATSUKA, Kobe-shi (JP); Shusuke CHINO, Tokyo (JP); Tsuyoshi NAGATA, Tokyo (JP); Yutaka KARASAWA, Tokyo (JP); Shinichiro SEO, Tokyo (JP)

(21) Appl. No.: 17/210,781

(22) Filed: Mar. 24, 2021

(30) Foreign Application Priority Data
Mar. 26, 2020 (JP) 2020-055965

(51) Int. Cl. A61G 13/12; A61B 34/30; A61B 34/20

(52) U.S. Cl. CPC A61G 13/1245; A61B 34/30; A61B 2090/3762; A61B 2034/303; A61B 34/20

(57) ABSTRACT

A surgical bed includes: a table on which a body part of a subject is placed; a leg holder configured to hold a leg part of the subject; a support member configured to connect the table and the leg holder to each other, and configured to adjustably support a positional relationship between the leg holder and the table; and a processor configured to derive the positional relationship between the leg holder and the table.

### CROSS-REFERENCE TO RELATED APPLICATIONS

This application is based on and claims priority under 35 USC 119 from Japanese Patent Application No. 2020-055965 filed on Mar. 26, 2020, the contents of which are incorporated herein by reference.

### TECHNICAL FIELD

The present disclosure relates to a surgical bed, an endoscopic surgical device, an endoscopic surgical method, and a system.

### BACKGROUND ART

In the related art, transanal minimally invasive surgery (TAMIS) is known as one of the surgical procedures. In TAMIS, it is known to install a platform (Transanal Access Platform) in an anus of a patient in order to insert a surgical instrument into the patient (refer to GelPOINT Path, Transanal Access Platform, Applied Medical, searched on Dec. 26, 2019, Internet <URL: https://www.appliedmedical.com/Products/Gelpoint/Path>).

In the related art, imaging with a CT scanner is performed before surgery, and volume data is acquired. A preoperative image is generated based on the volume data. In many cases, imaging by the CT scanner is performed with the subject in a supine position.

In TAMIS, in many cases, the body position is employed in which the waist is bent, such as a lithotomy position, knee-chest position, lateral recumbent position, or jackknife position.

Therefore, the body position when imaging the preoperative image and the body position during surgery differ from each other, the positions of the organs and bones which are surgery targets in the subject changes, and the surgical accuracy can decrease.

In view of the above-described circumstances, the present disclosure provides a surgical bed, a robotically-assisted surgical device, a robotically-assisted surgical method, and a system that can suppress a decrease in the surgical accuracy of robotic surgery even when the body position of the subject corresponding to the volume data obtained before surgery and the body position of the subject during surgery differ from each other.

### SUMMARY

A surgical bed related to one aspect of the present disclosure includes: a table on which a body part of a subject is placed; a leg holder configured to hold a leg part of the subject; a support member configured to connect the table and the leg holder to each other, and configured to adjustably support a positional relationship between the leg holder and the table; and a processor configured to derive the positional relationship between the leg holder and the table.

According to the present disclosure, even when the body position of the subject corresponding to the volume data obtained before surgery and the body position of the subject during surgery differ from each other, it is possible to suppress decrease in the surgical accuracy of robotic surgery.

### BRIEF DESCRIPTION OF DRAWINGS

Exemplary embodiment(s) of the present invention will be described in detail based on the following figures, wherein:

FIG. 1A is a block diagram illustrating a configuration example of a robotically-assisted surgical system according to a first embodiment;

FIG. 1B is a block diagram illustrating a hardware configuration example of a robotically-assisted surgical device;

FIG. 2A is a block diagram illustrating a functional configuration example of the robotically-assisted surgical device;

FIG. 2B is a block diagram illustrating a configuration example of a surgical bed;

FIG. 3 is a view illustrating an example of a platform, a surgical instrument, and an internal state of a subject;

FIG. 4 is a view illustrating an example of a kinematic model of a lower limb of the subject;

FIG. 5 is a view illustrating an example of a state of pelvis in a state where a body position of the subject is a lithotomy position and a leg part is raised low;

FIG. 6 is a view illustrating an example of a state of pelvis in a state where the body position of the subject is the lithotomy position and the leg part is raised high;

FIG. 7A is a side view of the surgical bed viewed from an x-direction;

FIG. 7B is a side view of the surgical bed viewed from a z-direction;

FIG. 7C is an upper view of the surgical bed viewed from a y-direction;

FIG. 7D is a side view of a base of the surgical bed in an extended state, viewed from the x-direction;

FIG. 7E is a side view of a table of the surgical bed in a state of being slid to a negative side in the z-direction, viewed from the x-direction;

FIG. 7F is a side view of the table of the surgical bed in a state of being slid to a positive side in the z-direction, viewed from the x-direction;

FIG. 7G is a side view of the table of the surgical bed in a state of being tilted with the positive side in the z-direction lowered, viewed from the x-direction;

FIG. 7H is a side view of the table of the surgical bed in a state of being tilted with the positive side in the z-direction raised, viewed from the x-direction;

FIG. 7I is a side view of the table of the surgical bed in a state of being tilted with the positive side in the x-direction lowered, viewed from the z-direction;

FIG. 7J is a side view of the table of the surgical bed in a state of being tilted with the positive side in the x-direction raised, viewed from the z-direction;

FIG. 7K is a top view of the surgical bed in a leg-opened state, viewed from the y-direction;

FIG. 7L is a top view of the surgical bed in a leg-closed state, viewed from the y-direction;

FIG. 7M is a top view of a state where a distance between the table of the surgical bed and a leg holding unit is increased, viewed from the x-direction;

FIG. 7N is a side view of a state where a support member of the surgical bed is tilted with respect to a horizontal direction, viewed from the x-direction;

FIG. 7O is a top view of a state where an angle of the leg holding unit of the surgical bed is adjusted, viewed from the x-direction;

FIG. 8 is a view illustrating an example of a form of the surgical bed corresponding to a jackknife position;

FIG. 9 is a flowchart illustrating an operation example of the robotically-assisted surgical device;

FIG. 10 is a flowchart illustrating an operation example of the robotically-assisted surgical device (continued from FIG. 9);

FIG. 11 is a view illustrating an example of a first display image; and

FIG. 12 is a side view illustrating a deformation configuration example of the surgical bed.

### DESCRIPTION OF EMBODIMENTS

Hereinafter, embodiments of the present disclosure will be described with reference to the drawings.

### First Embodiment

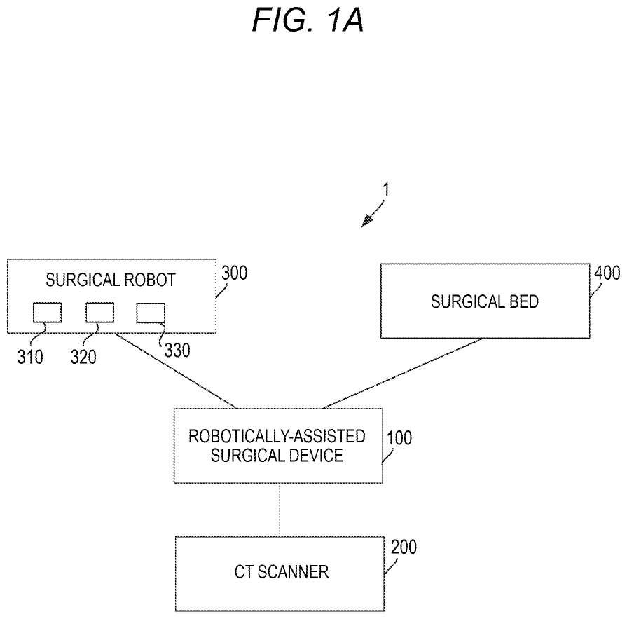

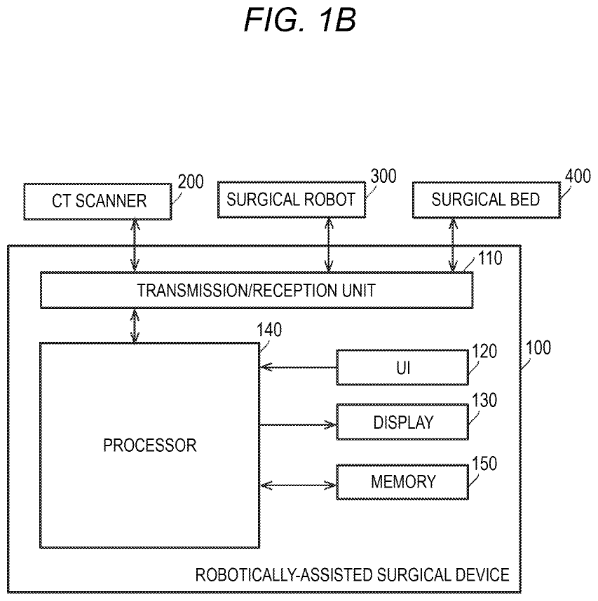

FIG. 1A is a block diagram illustrating a configuration example of a robotically-assisted surgical system 1 according to a first embodiment. The robotically-assisted surgical system 1 includes a robotically-assisted surgical device 100, a CT scanner 200, a surgical robot 300, and a surgical bed 400. The robotically-assisted surgical device 100, the CT scanner 200, the surgical robot 300, and the surgical bed 400 may be connected to each other via a network. The robotically-assisted surgical device 100 may be connected to each device of the CT scanner 200, the surgical robot 300, and the surgical bed 400 on a one-to-one basis. FIG. 1A exemplifies that the robotically-assisted surgical device 100 is connected to each of the CT scanner 200, the surgical robot 300, and the surgical bed 400.

The robotically-assisted surgical device 100 acquires various pieces of data from the CT scanner 200, the surgical robot 300, and the surgical bed 400. The robotically-assisted surgical device 100 performs image processing based on the acquired data to assist the robotic surgery by the surgical robot 300. The robotically-assisted surgical device 100 may be configured of a PC and software installed in the PC. The robotically-assisted surgical device 100 may be built into the surgical robot 300. The robotically-assisted surgical device 100 performs surgery navigation. The surgery navigation includes, for example, preoperative simulation for performing planning before surgery (preoperative planning) and intraoperative navigation for performing the assistance during surgery.

The CT scanner 200 irradiates the subject with X-rays, and captures images (CT images) by using the difference in X-ray absorption by tissues in the body. The subject may include a living body, a human body, an animal, and the like. The CT scanner 200 generates the volume data including information on any location on the inside of the subject. The CT scanner 200 transmits the volume data as the CT image to the robotically-assisted surgical device 100 via a wired circuit or a wireless circuit. Imaging conditions for CT images or contrast conditions for administration of a contrast medium may be taken into consideration when capturing CT images.

The surgical robot 300 includes a robot operation terminal 310, a robot main body 320, and an image display terminal 330.

The robot operation terminal 310 includes a hand controller and a foot switch operated by an operator. The robot operation terminal 310 operates a plurality of robot arms AR provided in the robot main body 320 according to the operation of the hand controller or the foot switch by the operator. The robot operation terminal 310 includes a viewer. The viewer may be a stereo viewer, and may display a three-dimensional image by fusing the images captured by an endoscope ES (endoscope camera). The plurality of robot operation terminals 310 may exist, and the robotic surgery may be performed by a plurality of operators operating the plurality of robot operation terminals 310.

The robot main body 320 includes the plurality of robot arms AR for performing the robotic surgery, an end effector EF (forceps, instruments) attached to the robot arm AR, and the endoscope ES attached to the robot arm AR. Since the end effector EF and the endoscope ES are equivalent to those used for endoscopic surgery, the end effector EF and the endoscope ES are also referred to as surgical instruments 30 in the embodiment. The surgical instrument 30 includes at least one of one or more end effectors EF and endoscopes ES.

The robot main body 320 is provided with, for example, four robot arms AR, and includes a camera arm to which the endoscope ES is attached, a first end effector arm to which the end effector EF operated by the hand controller for the right hand of the robot operation terminal 310 is attached, a second end effector arm to which the end effector EF operated by the hand controller for the left hand of the robot operation terminal 310 is attached, and a third end effector arm to which the end effector EF for the replacement is attached. Each robot arm AR has a plurality of joints, and may be provided with a motor and an encoder corresponding to each joint. The encoder may include a rotary encoder as an example of an angle detector. Each robot arm AR has at least 6 degrees of freedom, preferably 7 or 8 degrees of freedom, and may operate in the three-dimensional space and be movable in each direction within the three-dimensional space. The end effector EF is an instrument that actually comes into contact with the treatment target in a subject PS in the robotic surgery, and enables various treatments (for example, grasping, excision, peeling, and suturing).

The end effector EF may include, for example, grasping forceps, peeling forceps, an electric knife, and the like. As the end effector EF, a plurality of separate end effector EFs different for each role may be prepared. For example, in the robotic surgery, the tissue may be suppressed or pulled by two end effector EFs, and the tissue may be cut by one end effector EF. The robot arm AR and the surgical instrument 30 may operate based on an instruction from the robot operation terminal 310. At least two end effectors EF are used in the robotic surgery.

The image display terminal 330 has a monitor and a controller for processing the image captured by the endoscope ES and displaying the image on a viewer or a monitor. The monitor is confirmed by, for example, a robotic surgery assistant or a nurse.

The surgical robot 300 performs the robotic surgery in which an operation of the hand controller or the foot switch of the robot operation terminal 310 by the operator is received, the operations of the robot arm AR, the end effector EF, and the endoscope ES of the robot main body 320 are controlled, and various treatments for the subject PS are performed. In the robotic surgery, the endoscopic surgery may be performed in the subject PS.

In the embodiment, it is mainly assumed that the transanal minimally invasive surgery (TAMIS) is performed using the surgical robot 300. In TAMIS, a platform 40 (Transanal Access Platform) is installed on the anus of the subject PS in order to insert the surgical instrument 30 into the subject PS. In TAMIS, since the platform 40 is installed on the anus, which is a hole of the subject PS, it is not necessary to perforate a port on the body surface of the subject PS unlike installation of a trocar. In TAMIS, gas may be injected through the platform 40 to inflate the tissues or organs existing in the neighborhood of the anus of the subject PS. The tissues or organs existing in the neighborhood of the anus of the subject PS may include, for example, a rectum, colon, prostate, and the like. The platform 40 has a valve and maintains the inside of the subject PS airtight. Air (for example, carbon dioxide) may be continuously introduced into the subject PS for maintaining the airtight state.

The surgical instrument 30 is inserted into the platform 40. The valve of the platform 40 is opened when the surgical instrument 30 is inserted, and the valve of the platform 40 is closed when the surgical instrument 30 is detached. The surgical instrument 30 is inserted via the platform 40, and various treatments are performed depending on the surgical procedure. The robotic surgery may be applied to the endoscopic surgery (for example, palatal jaw surgery or mediastinal surgery) of other parts in addition to a case where the organs neighbor of the anus are surgery targets.

The platform 40 is installed on the anus, but the access platform for single incisional laparoscopic surgery (SILS) can be used as it is or with some modification. The platform 40 may be a platform dedicated to the anus. As for the surgical robot, a surgical robot for laparoscopic surgery can be used as the surgical robot 300 for the anus of the embodiment.

FIG. 1B is a block diagram illustrating a configuration example of the robotically-assisted surgical device 100. The robotically-assisted surgical device 100 includes a transmission/reception unit 110, a UI 120, a display 130, a processor 140, and a memory 150.

The transmission/reception unit 110 includes a communication port, an external device connection port, a connection port to an embedded device, and the like. The transmission/reception unit 110 acquires various pieces of data from the CT scanner 200, the surgical robot 300, and the surgical bed 400. The various pieces of acquired data may be immediately sent to the processor 140 (a processing unit 160) for various types of processing, or may be sent to the processor 140 for various types of processing when necessary after being stored in the memory 150. The various pieces of data may be acquired via a recording medium or a storage medium.

The transmission/reception unit 110 transmits and receives various pieces of data to and from the CT scanner 200, the surgical robot 300, and the surgical bed 400. The various pieces of data to be transmitted may be directly transmitted from the processor 140 (the processing unit 160), or may be transmitted to each device when necessary after being stored in the memory 150. The various pieces of data may be sent via a recording medium or a storage medium.

The transmission/reception unit 110 may acquire volume data from the CT scanner 200. The volume data may be acquired in the form of intermediate data, compressed data or sinogram. The volume data may be acquired from information from a sensor device attached to the robotically-assisted surgical device 100.

The transmission/reception unit 110 acquires information from the surgical robot 300. The information from the surgical robot 300 may include information on the kinematics of the surgical robot 300. The information on the kinematics may include, for example, shape information regarding the shape and motion information regarding the operating range of an instrument (for example, the robot arm AR, the end effector EF, the endoscope ES) for performing the robotic surgery included in the surgical robot 300. The information on the kinematics may be received from an external server.

The shape information may include at least a part of information such as the length and weight of each part of the robot arm AR, the end effector EF, and the endoscope ES, the angle of the robot arm AR with respect to the reference direction (for example, a horizontal surface), and the attachment angle of the end effector EF with respect to the robot arm AR.

The motion information may include the movable range in the three-dimensional space of the robot arm AR, the end effector EF, and the endoscope ES. The motion information may include information such as the position, speed, acceleration, or orientation of the robot arm AR when the robot arm AR operates. The motion information may include information such as the position, speed, acceleration, or orientation of the end effector EF with respect to the robot arm AR when the end effector EF operates. The motion information may include information such as the position, speed, acceleration, or orientation of the endoscope ES with respect to the robot arm AR when the endoscope ES operates.

An angle sensor may be attached to the robot arm AR, the end effector EF, or the endoscope ES. The angle sensor may include a rotary encoder that detects an angle corresponding to the orientation of the robot arm AR, the end effector EF, or the endoscope ES in a three-dimensional space. The transmission/reception unit 110 may acquire the detection information detected by various sensors attached to the surgical robot 300.

The transmission/reception unit 110 may acquire operation information regarding the operation with respect to the robot operation terminal 310. The operation information may include information such as an operation target (for example, the robot arm AR, the end effector EF, the endoscope ES), an operation type (for example, movement, rotation), an operation position, and an operation speed.

The transmission/reception unit 110 may acquire surgical instrument information regarding the surgical instrument 30. The surgical instrument information may include the insertion distance of the surgical instrument 30 to the subject PS. The insertion distance corresponds, for example, to the distance between the platform 40 into which the surgical instrument 30 is inserted and the distal end position of the surgical instrument 30. For example, the surgical instrument 30 may be provided with a scale indicating the insertion distance of the surgical instrument 30. The transmission/reception unit 110 may electronically read the scale to obtain the insertion distance of the surgical instrument 30. In this case, for example, a linear encoder (reading device) may be attached to the platform 40, and the surgical instrument 30 may be provided with an encoding marker. The transmission/reception unit 110 may acquire the insertion distance of the surgical instrument 30 as the operator reads the scale and inputs the insertion distance via the UI 120.

The information from the surgical robot 300 may include information regarding the imaging by the endoscope ES (endoscopic information). The endoscopic information may include an image captured by the endoscope ES (actual endoscopic image) and additional information regarding the actual endoscopic image (imaging position, imaging orientation, imaging viewing angle, imaging range, imaging time, and the like).

The transmission/reception unit 110 may acquire information from the surgical bed 400. The information from the surgical bed 400 may include detection information by various sensors SR included in the surgical bed 400, information derived by the surgical bed 400 (for example, information on the positional relationship of each unit in the surgical bed 400), and the like.

The UI 120 may include, for example, a touch panel, a pointing device, a keyboard, or a microphone. The UI 120 receives any input operation from the operator of the robotically-assisted surgical device 100. Operators may include doctors, nurses, radiologists, students, and the like.

The UI 120 receives various operations. For example, an operation, such as designation of a region of interest (ROI) or setting of a brightness condition (for example, window width (WW) or window level (WL)), in the volume data or in an image (for example, a three-dimensional image or a two-dimensional image which will be described later) based on the volume data, is received. The ROI may include regions of various tissues (for example, blood vessels, organs, viscera, bones, and brain). The tissue may include diseased tissue, normal tissue, tumor tissue, and the like.

The display 130 may include an LCD, for example, and displays various pieces of information. The various pieces of information may include a three-dimensional image and a two-dimensional image obtained from the volume data. The three-dimensional images may include a volume rendering image, a surface rendering image, a virtual endoscopic image, a virtual ultrasound image, a CPR image, and the like. The volume rendering images may include a RaySum image, an MIP image, an MinIP image, an average value image, a raycast image, and the like. The two-dimensional images may include an axial image, a sagittal image, a coronal image, an MPR image, and the like, or may include a synthetic image of theses images.

The memory 150 includes various primary storage devices such as ROM and RAM. The memory 150 may include a secondary storage device such as HDD or SSD. The memory 150 may include a tertiary storage device such as a USB memory, an SD card, or an optical disk. The memory 150 stores various pieces of information and programs. The various pieces of information may include volume data acquired by the transmission/reception unit 110, images generated by the processor 140, setting information set by the processor 140, and various programs. The memory 150 is an example of a non-transitory recording medium in which a program is recorded.

The processor 140 may include a CPU, a DSP, or a GPU. The processor 140 functions as the processing unit 160 that performs various types of processing and controls by executing the program stored in the memory 150.

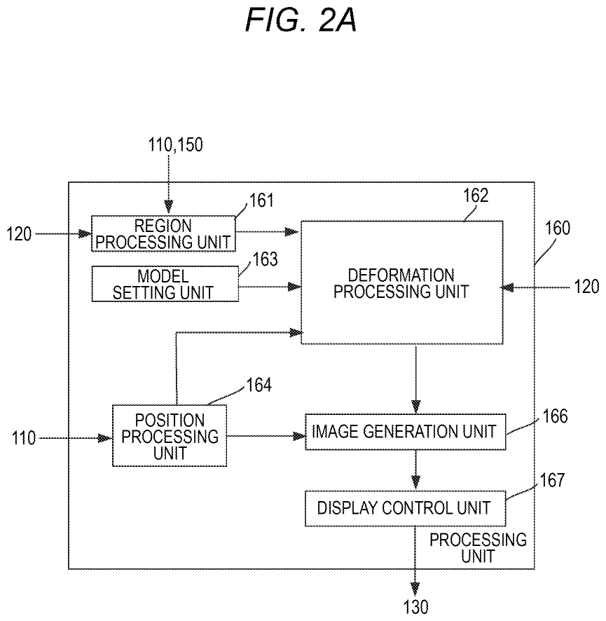

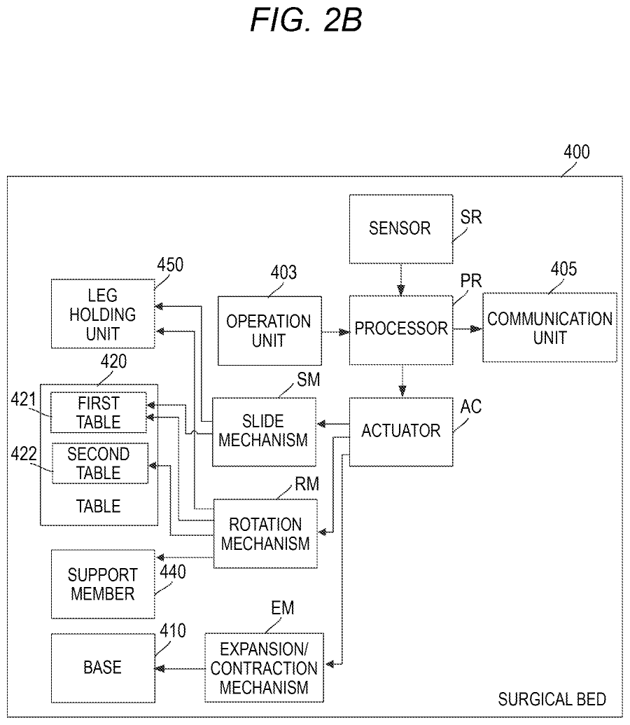

FIG. 2A is a block diagram illustrating a functional configuration example of the processing unit 160. The processing unit 160 includes a region processing unit 161, a deformation processing unit 162, a model setting unit 163, a position processing unit 164, an image generation unit 166, and a display control unit 167. Each unit included in the processing unit 160 may be realized as different functions by one piece of hardware, or may be realized as different functions by a plurality of pieces of hardware. Each unit included in the processing unit 160 may be realized by a dedicated hardware component.

The region processing unit 161 acquires the volume data of the subject via the transmission/reception unit 110, for example. The region processing unit 161 extracts any region included in the volume data. The region processing unit 161 may automatically designate the ROI and extract the ROI based on a pixel value of the volume data, for example. The region processing unit 161 may manually designate the ROI and extract the ROI via the UI 120, for example. The ROI may include regions such as organs, bones, blood vessels, affected parts (for example, diseased tissue or tumor tissue). Organs may include rectum, colon, prostate, and the like.

The ROI may be segmented (divided) and extracted including not only a single tissue but also tissues around the tissue. For example, in a case where the organ which is the ROI is the rectum, not only the rectum itself, but also blood vessels that are connected to the rectum or run in or in the neighborhood of the rectum, bones (for example, spine, pelvis) or muscles neighbor of the rectum, may also be included. The above-described rectum itself, the blood vessels in or in the neighborhood of the rectum, and the bones or muscles neighbor of the rectum may be segmented and obtained as separate tissues.

The model setting unit 163 sets a model of the tissue. The model may be set based on the ROI and the volume data. The model visualizes the tissue visualized by the volume data in a simpler manner than the volume data. Therefore, the data amount of the model is smaller than the data amount of the volume data corresponding to the model. The model is a target of deformation processing and deforming operation imitating various treatments in surgery, for example. The model may be, for example, a bone deformation model. In this case, the model deforms the bone by assuming a frame in a simple finite element and moving the vertices of the finite element. The deformation of the tissue can be visualized by following the deformation of the bone. The model may include an organ model imitating an organ (for example, rectum). The model may have a shape similar to a simple polygon (for example, a triangle), or may have other shapes. The model may be, for example, a contour line of the volume data indicating an organ. The model may be a three-dimensional model or a two-dimensional model.

The model setting unit 163 may generate a kinematic model (will be described below) for the bones and joints of the lower limb of the subject PS. In the deformation of the kinematics, the bone may be visualized by the deformation of the volume data instead of the deformation of the model. This is because, since the bone has a low degree of freedom of deformation, visualization is possible by affine transformation of the volume data. The model setting unit 163 may subordinate bone deformation models or finite element models of tissues other than bones to the kinematic model. Subordinating bone deformation models or finite element models of tissues other than bones to the kinematic model may mean that organs are hanging from the bone.

The model setting unit 163 may acquire the model by generating the model based on the volume data. A plurality of model templates may be predetermined and stored in the memory 150 or an external server. The model setting unit 163 may acquire a model by acquiring one model template among a plurality of model templates prepared in advance from the memory 150 or the external server in accordance with the volume data.

The model setting unit 163 may set the position of a target TG in the tissue (for example, rectum) of the subject PS included in the volume data. The model setting unit 163 may set the position of the target TG in the organ model. The target TG is set in any tissue. The model setting unit 163 may designate the position of the target TG via the UI 120. The position of the target TG (for example, affected part) treated in the past for the subject PS may be stored in the memory 150. The model setting unit 163 may acquire and set the position of the target TG from the memory 150. The model setting unit 163 may set the position of the target TG depending on the surgical procedure. The surgical procedure indicates a method of surgery for the subject PS. The target position may be the position of the region of the target TG having a certain size.

The deformation processing unit 162 performs processing related to the deformation in the subject PS which is a surgery target. For example, the tissue of an organ or the like in the subject PS can be subjected to various deforming operations by the operator by imitating various treatments performed by the operator in surgery. The deforming operation may include an operation of lifting an organ, an operation of flipping an organ, an operation of cutting an organ, and the like. Correspondingly, the deformation processing unit 162 deforms the organ model. For example, an organ can be pulled, pushed, or cut by the end effector EF, but this state may be simulated by deforming the organ model in this manner. When the organ model deforms, the targets TG in the model may also deform.

The deformation by the deforming operation may be performed with respect to the model and may be a large deformation simulation using the finite element method. For example, movement of organs or movement of bones of lower limbs due to the body position change may be simulated. In this case, the elastic force applied to the contact point of organs, diseases, or bones of lower limbs, the rigidity of organs, diseases, or bones of lower limbs, and other physical characteristics may be taken into consideration. The movement of bones may be visualized by the kinematic model. In the deformation processing with respect to the model, the computation amount is reduced as compared with the deformation processing with respect to the volume data. This is because the number of elements in the deformation simulation is reduced. The deformation processing with respect to the model may not be performed, and the deformation processing may be directly performed with respect to the volume data.

The deformation processing unit 162 may perform the gas injection simulation in which gas is virtually injected into the subject PS through the anus, for example, as processing related to the deformation. The specific method of the gas injection simulation may be a known method, and for example, a pneumoperitoneum simulation method described in Reference Non-Patent Literature 1 (Takayuki Kitasaka, Kensaku Mori, Yuichiro Hayashi, Yasuhito Suenaga, Makoto Hashizume, and Jun-ichiro Toriwaki, “Virtual Pneumoperitoneum for Generating Virtual Laparoscopic Views Based on Volumetric Deformation”, MICCAI (Medical Image Computing and Computer-Assisted Intervention), 2004, P 559-P 567) may be applied to the gas injection simulation in which gas is injected through the anus.

In other words, the deformation processing unit 162 may perform the gas injection simulation based on the model of the non-gas injection state or the volume data, and generate the model of the virtual gas injection state or the volume data. By the gas injection simulation, the operator can observe the state where gas is virtually injected by assuming that the subject PS is in a state where gas is injected without actually injecting gas into the subject PS. Of the gas injection states, a gas injection state estimated by the gas injection simulation may be referred to as a virtual gas injection state, and a state where gas is actually injected may be referred to as an actual gas injection state.

The gas injection simulation may be a large deformation simulation using the finite element method. In this case, the deformation processing unit 162 may segment the body surface containing the subcutaneous fat of the subject PS and an internal organ near the anus of the subject PS, via the region processing unit 161. The deformation processing unit 162 may model the body surface as a two-layer finite element of skin and body fat, and model the internal organ near the anus as a finite element, via the model setting unit 163. The deformation processing unit 162 may segment, for example, the rectum and bones in any manner, and add the segmented result to the model. A gas region may be provided between the body surface and the internal organ near the anus, and the gas injection region may be expanded (swollen) in response to the virtual gas injection. The gas injection simulation may not be performed.

The position processing unit 164 acquires information on the positional relationship between predetermined positions on the surgical bed 400 from the surgical bed 400 via the transmission/reception unit 110. The information on the positional relationship includes the information on the positional relationship between a leg holding unit 450 and a table 420 of the surgical bed 400, which will be described later. The leg holding unit 450 corresponds to a leg holder in the present disclosure.

The position processing unit 164 acquires 3D data of the subject PS. The 3D data may include the volume data or the model of the subject PS. The volume data or the model may be in the non-gas injection state or the gas injection state. The 3D data may be surface data generated from the volume data.

The position processing unit 164 estimates at least a state of a pelvis 14 of the subject PS based on the acquired 3D data and the positional relationship between the leg holding unit 450 and the table 420. The state of the pelvis 14 may include the position, orientation or movement of the pelvis 14 in the 3D data. The position processing unit 164 calculates the deformation of a pelvic organ 51 in the 3D data based on the state of the pelvis 14. The pelvic organs 51 are organs that at least partially exist inside the pelvis 14, and includes the rectum, colon, prostate, and the like. The position processing unit 164 may estimate the state (for example, position or slope) of bones (for example, femur 13 or spine) other than the pelvis 14.

The position processing unit 164 may acquire the position and orientation of the surgical instrument 30. The surgical bed 400 and the surgical robot 300 may be arranged in a predetermined positional relationship. The subject PS is placed in a predetermined position and fixed to the surgical bed 400. Therefore, the position and angle of the surgical instrument 30 may be estimated based on the kinematics of the surgical robot 300 and the angle of the robot arm AR. The position of the surgical instrument 30 may be calculated based on the position processing unit 164 and the insertion distance of the surgical instrument 30. Accordingly, the position processing unit 164 can derive the position and orientation in the 3D data.

The image generation unit 166 generates various images. The image generation unit 166 generates a three-dimensional image or a two-dimensional image based on at least a part of the acquired volume data (for example, a region extracted in the volume data). The image generation unit 166 may generate a three-dimensional image or a two-dimensional image based on the volume data deformed by the deformation processing unit 162. For example, a volume rendering image or a virtual endoscopic image visualizing a state where the orientation of the endoscope ES is viewed from the position of the endoscope ES may be generated.

The display control unit 167 causes the display 130 to display various types of data, information, and images. The display control unit 167 displays an image (for example, a rendering image) generated by the image generation unit 166. The display control unit 167 superimposes and displays various pieces of information together with the image. The superimposed and displayed information may include, for example, information indicating the surgical instrument 30, the pelvis 14, and the pelvic organ 51. The display control unit 167 may also adjust the brightness of the rendering image. The brightness adjustment may include, for example, adjustment of at least one of a window width (WW) and a window level (WL).

FIG. 2B is a block diagram illustrating a configuration example of the surgical bed 400. The surgical bed 400 includes a base 410, a table 420, a support member 440, a leg holding unit 450, a processor PR, an actuator AC, an expansion/contraction mechanism EM, a rotation mechanism RM, a slide mechanism SM, and a sensor SR. The surgical bed 400 also includes an operation unit 403 and a communication unit 405. There may be a plurality of actuators AC, which are illustrated as actuators AC0, AC1, AC2, and . . . . There may be a plurality of rotation mechanisms RM, which are illustrated as rotation mechanisms RM1, RM2, and . . . . There may be a plurality of slide mechanisms SM, which are illustrated as slide mechanisms SM1, SM2, and . . . . There can be a plurality of sensors SR, which are illustrated as sensors SR0, SR1, SR2, and . . . . The sensor SR may also be built into each mechanism or actuator AC as an encoder or the like. The sensor SR may be built only into the leg holding unit 450, and may contain information (distance, angle, rotation) that can specify the position with respect to the table 420.

The actuator AC provides a driving force to the rotation mechanism RM and the slide mechanism SM, according to the control by the processor PR.

The processor PR may include a CPU, a DSP, or a MPU. The processor PR functions as a processing unit that performs various processing and control by executing a program stored in the memory provided by the surgical bed 400.

For example, the processor PR may acquire the operation information by the operator of the surgical bed 400 acquired via the operation unit 403, and control the actuator AC based on the operation information. The operation information may include, for example, operation information for designating the position of the leg holding unit 450 for raising and lowering a leg part 31, and operation information for designating the body position of the subject PS. Accordingly, the surgical bed 400 can manually change the form of the surgical bed 400 to assist the subject PS in any body position. The posture of the subject PS can be easily changed to a posture that is easier for the operator to perform the procedure.

The processor PR may also acquire information on the body position of the subject PS via the communication unit 405 and control the actuator AC based on the body position of the subject PS. In this case, the actuator AC may provide the driving force necessary for the operations of the rotation mechanism RM and the slide mechanism SM, which are necessary for the acquired body position of the subject PS. Accordingly, the surgical bed 400 can easily and quickly correspond to the change of the body position of the subject PS to the body position required by the surgical robot 300 in the robotic surgery.

The processor PR may acquire information on the surgical procedure for the subject PS via the communication unit 405 and control the actuator AC based on the surgical procedure. In this case, the actuator AC may provide the driving force necessary for the operations of the rotation mechanism RM and the slide mechanism SM, which are necessary for setting the acquired body position for performing various treatments in the surgical procedure. Accordingly, the surgical bed 400 can easily and quickly correspond to the change of the body position of the subject PS to the body position required by the surgical robot 300 for the surgical procedure or various treatments of robotic surgery.

The processor PR may also acquire information on the position of the surgical instrument 30 in the subject PS via the communication unit 405 and control the actuator AC based on the position of the surgical instrument 30 in the subject PS. The information on the position of the surgical instrument 30 may be included in the information on the kinematics of the surgical robot 300. In this case, the actuator AC may provide the driving force necessary for the operation of the rotation mechanism RM and the slide mechanism SM for bringing the target TG of the subject PS closer to the position of the surgical instrument 30. Accordingly, the surgical bed 400 can easily and quickly change the body position of the subject PS such that the surgical robot 300 easily approaches the target TG using the surgical instrument 30.

In this manner, the form of the surgical bed 400 can be flexibly changed in conjunction with the surgical robot 300, and the surgical bed 400 can assist the smooth changing of the body position of the subject PS. By changing the form of the surgical bed 400, the surgical bed 400 can make it easy to approach tissues positioned behind bones and organs, for example, by taking into account the movement of the pelvis 14 or the pelvic organ 51 due to the action of gravity. The surgical bed 400 may also change the body position of the subject PS to adjust the blood stream of the subject PS.

The rotation mechanism RM receives the driving force from the actuator AC and rotates. The slide mechanism SM receives the driving force from the actuator AC to slide (move in parallel). The surgical bed 400 can finely adjust the body position of the subject PS without manual intervention during surgery, as the actuator AC provides the driving force to the rotation mechanism RM and the slide mechanism SM at the desired timing.

The sensors SR include position detectors (for example, linear encoders), angle detectors (for example, rotary encoders), and the like. The sensor SR may iteratively detect the position and angle of each unit in the surgical bed 400, and detect the movement of each unit in the surgical bed 400.

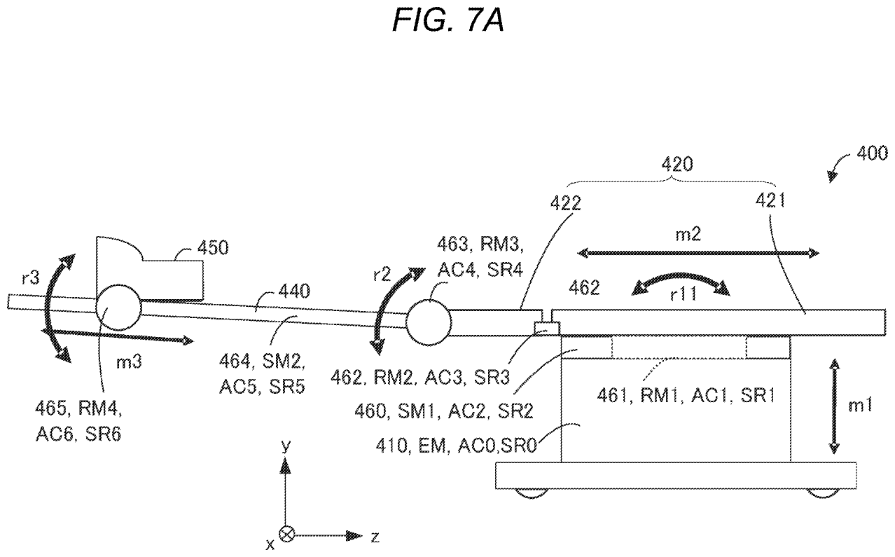

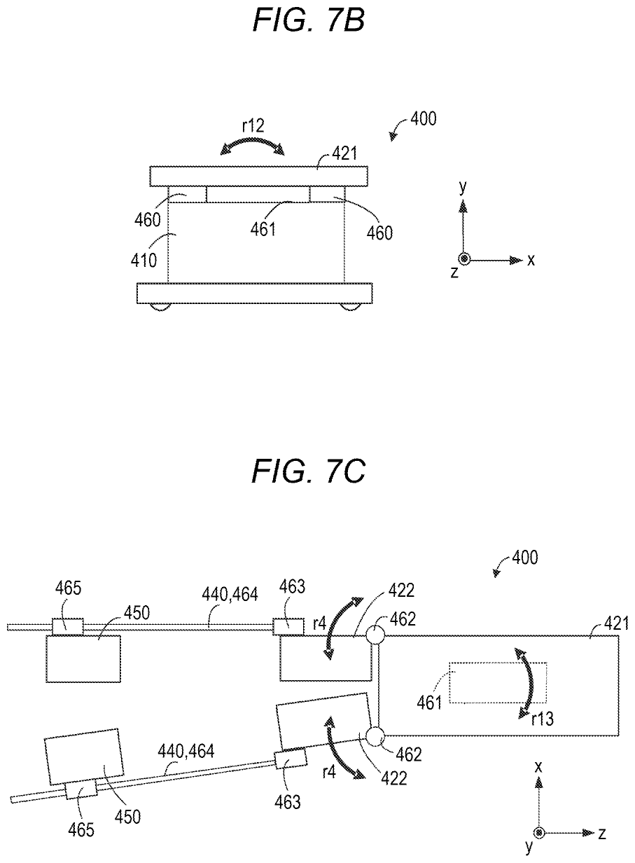

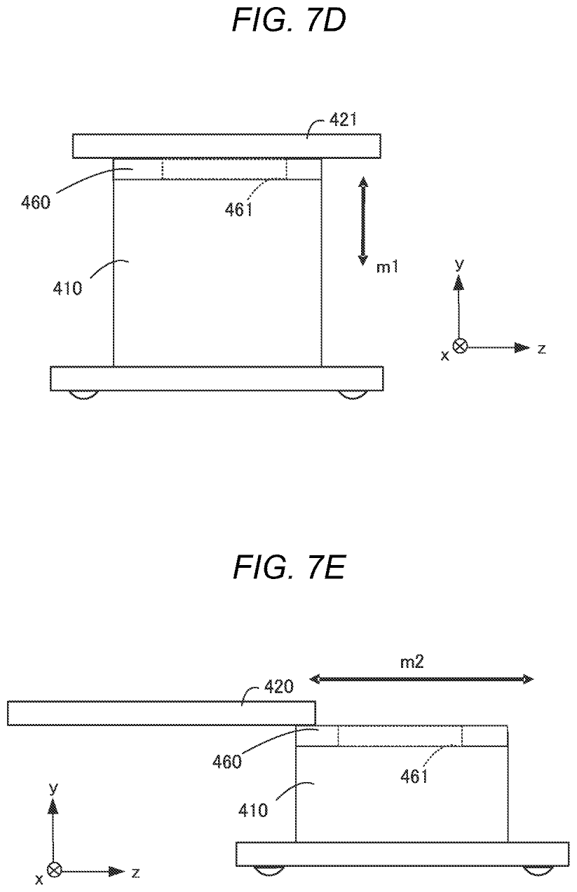

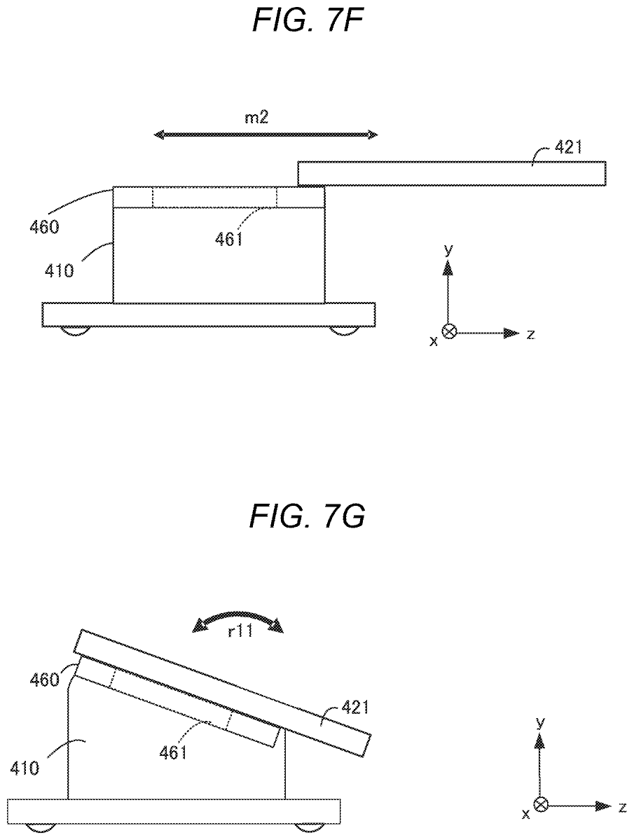

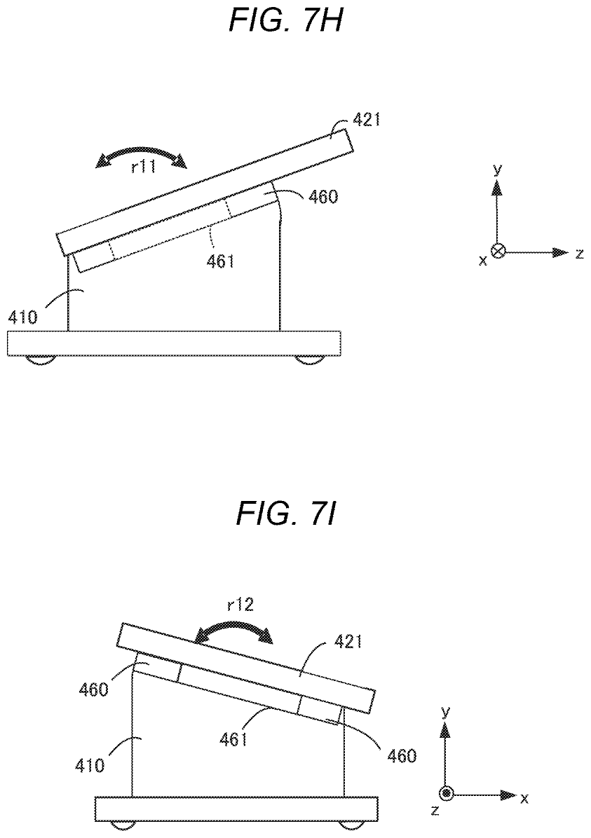

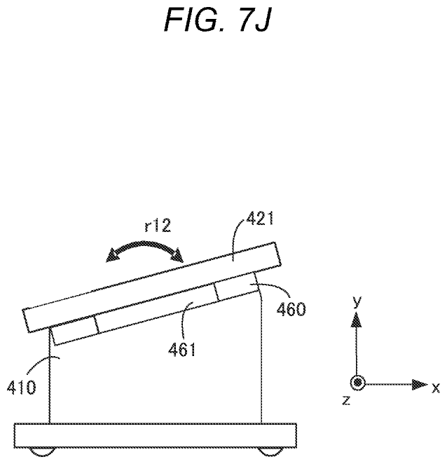

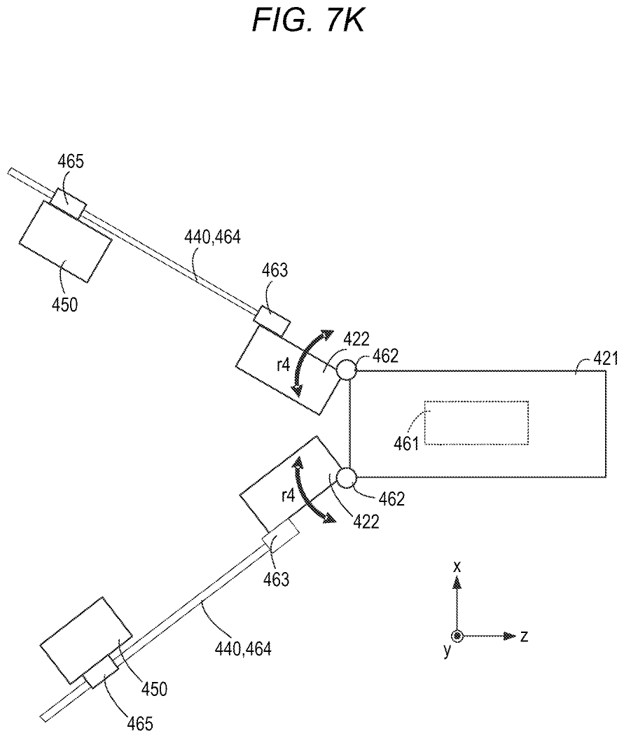

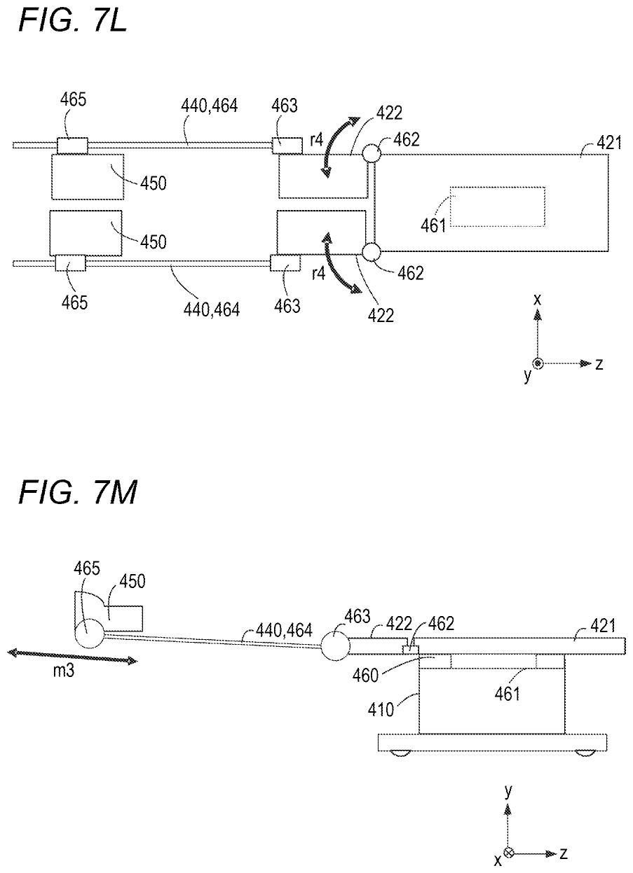

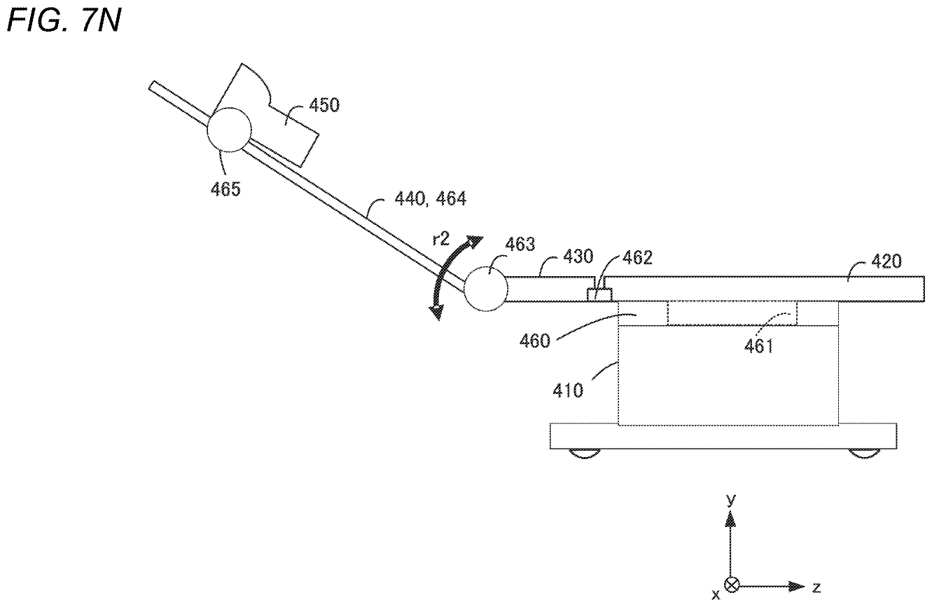

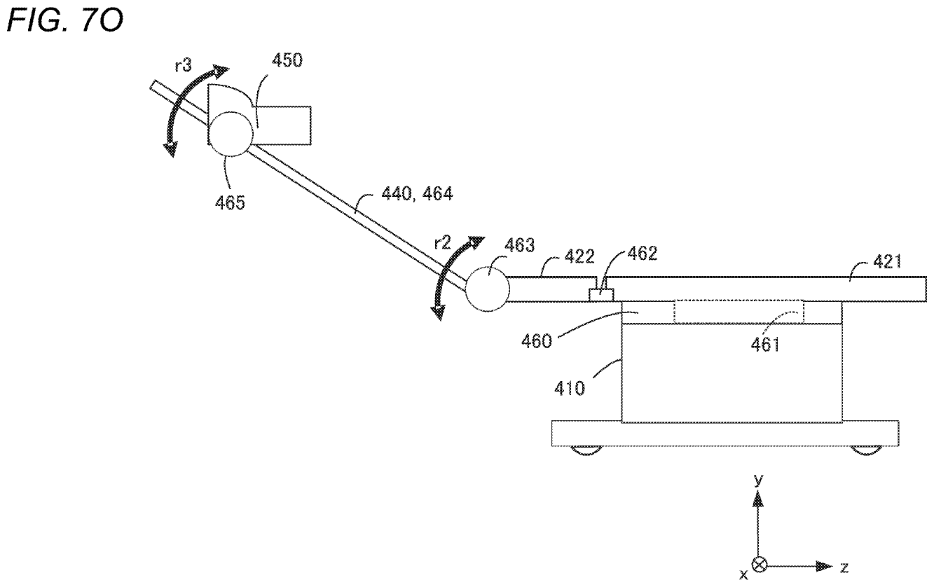

The base 410 (refer to FIG. 7A and the like) is arranged on the floor or the like of the operating room and holds the table 420. The base 410 can be expanded and contracted (raised and lowered) in the vertical direction according to the driving force from the actuator AC.

The table 420 (refer to FIG. 7A and the like) has a first table 421 and a second table 422. A body part 33 of the subject PS may be placed on the first table 421. A thigh part 32 of the subject PS may be placed on the second table 422. The first table 421 is rotatable with respect to the base 410 following the rotation of the rotation mechanism RM. The first table 421 may be rotatable, for example, with the directions of three axes mutually orthogonal to each other as the rotation centers. The first table 421 is movable along the surface of the table 420 following the slide of the slide mechanism SM. The second table 422 is rotatable with respect to the first table 421 following the rotation of the rotation mechanism RM.

The support member 440 (refer to FIG. 7A and the like) is connected to the table 420 and the leg holding unit 450. The support member 440 adjustably supports the angle of the leg holding unit 450 with respect to the table 420 and the distance between the table 420 and the leg holding unit 450. The support member 440 is rotatable with respect to the second table 422 following the rotation of the rotation mechanism RM.

The leg holding unit 450 (refer to FIG. 7A and the like) holds the leg part 31 of the subject PS. The leg holding unit 450 may hold the leg part 31 in a state where the thigh part 32 of the subject PS is bent. The leg holding unit 450 is rotatable with respect to the support member 440 following the rotation of the rotation mechanism RM. The leg holding unit 450 is movable along the extending direction of the support member 440 following the slide of the slide mechanism SM. The leg holding unit 450 is capable of towing the lower limb including the leg part 31 by rotatably and movably holding the leg part 31 of the subject PS. In other words, the leg holding unit 450 is an example of a lower limb towing device that tows the lower limb of the subject PS.

The processor PR cooperates with the sensor SR to derive the positional relationships of the plurality of parts in the surgical bed 400. For example, the processor PR derives the positional relationship between the leg holding unit 450 and the table 420. The positional relationship may be the position of the leg holding unit 450 with respect to the table 420, or the position of the table 420 with respect to the leg holding unit 450. For example, the processor PR may calculate the position of the leg holding unit 450 with respect to the table 420 based on the position and angle of each unit measured by the linear encoder and rotary encoder. The processor may also take into account the slope of the body of the surgical bed 400 to derive the above-described positional relationship. The slope of the body of the surgical bed 400 may be indicated by the slope of the base 410 with respect to the directions of three axes. The slope of the body of the surgical bed 400 is more likely to affect the movement of organs (for example, the pelvic organ 51) inside the subject PS than bones such as the pelvis 14.

The processor PR may obtain the detection result by the sensor SR iteratively and derive the positional relationship in a case where the detection result by the sensor SR changes. For example, the processor PR may calculate the positional relationship between the leg holding unit 450 and the table 420 in a case where it is detected that the leg holding unit 450 has moved along the support member 440 based on the detection value of the sensor SR.

The communication unit 405 may communicate various pieces of data with an external device (for example, the robotically-assisted surgical device 100). The various pieces of data include detection information detected by various sensors SR, information on the positional relationship between the leg holding unit 450 and the table 420, and the like.

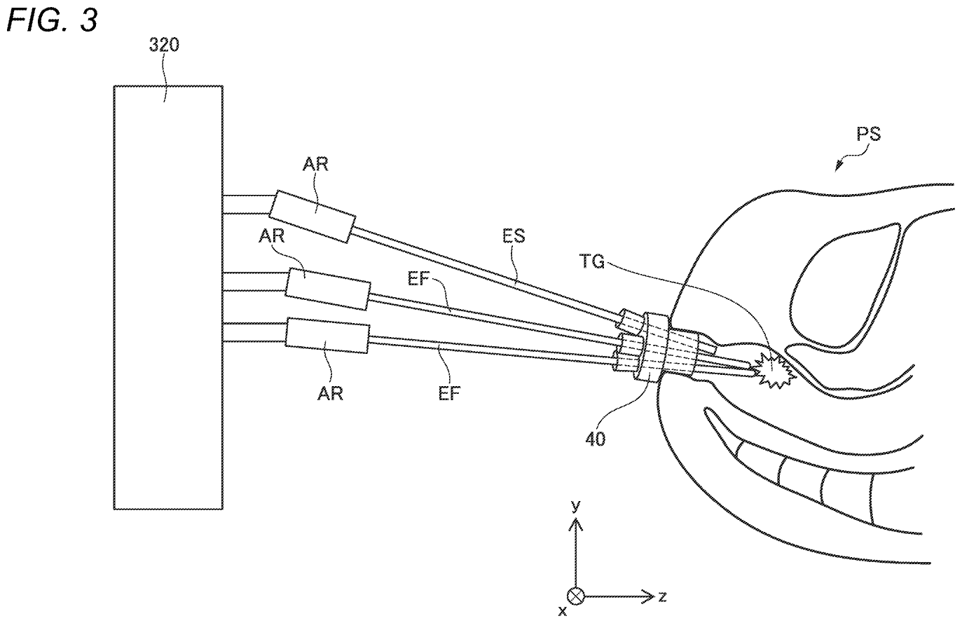

FIG. 3 is a view illustrating an example of the platform 40, the surgical instrument 30, and an internal state of the subject PS. The end effector EF attached to the robot arm AR of the robot main body 320 is inserted into the subject PS through the platform 40. In FIG. 3, the platform 40 is installed on the anus, and reaches the target TG where the disease exists at a part of the rectum connected to the anus and the treatment is performed. The state near the target TG is imaged by the endoscope ES attached to the robot arm AR. Similar to the end effector EF, the endoscope ES is also inserted into the subject PS through the platform 40.

In FIG. 3, the x-direction, the y-direction, and the z-direction of the subject coordinate system (patient coordinate system) with respect to the subject PS are illustrated. The subject coordinate system is an orthogonal coordinate system. The x-direction may be along the left-right direction with respect to the subject PS. The y-direction may be the front-rear direction (thickness direction of the subject PS) with respect to the subject PS. The z-direction may be an up-down direction (the body axial direction of the subject PS) with respect to the subject PS. The x-direction, the y-direction, and the z-direction may be three directions defined by digital imaging and communications in medicine (DICOM). The coordinate system is the same in the following drawings.

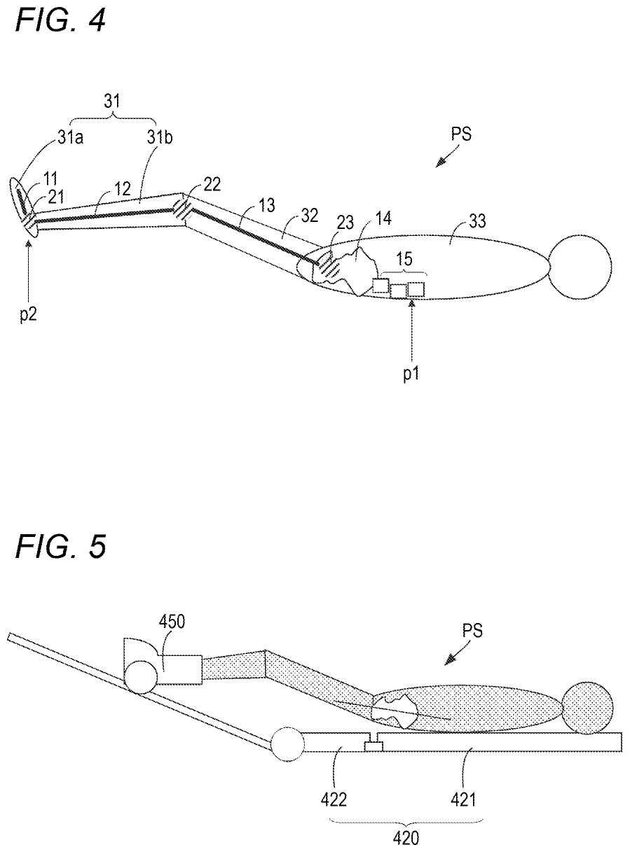

FIG. 4 is a view illustrating an example of the kinematic model of a lower limb of the subject PS. The kinematic model has information on the length of the bones in the lower limb and the degrees of freedom of the joints in the lower limb. The lower limb may be, for example, a part closer to the distal end side of the foot than the near part of the lumbar vertebrae. The lower limb of the subject PS has the leg part 31, the thigh part 32, and the body part 33. The leg part 31 has a foot part 31a and a shin part 31b. An ankle joint 21 is interposed between a tarsus 11 as a bone of the foot part 31a and a tibia 12 of the shin part 31b. A knee joint 22 is interposed between the tibia 12 and the femur 13 of the thigh part 32. A hip joint 23 is interposed between the femur 13 and the pelvis 14 of the body part 33. On the head part side, a lumbar vertebrae 15 is connected to the hip joint 23.

The model setting unit 163 generates a kinematic model based on the 3D data of the subject PS. In this case, for example, the region processing unit 161 acquires both end parts of the region of the bones of the lower limb of the subject PS designated in the 3D data via the UI 120, and extracts the region of the bones positioned between the two end parts. The two end parts of the designated region of bones on the lower limb side may be the tarsus 11 and the lumbar vertebrae 15 (for example, the bone near a first lumbar vertebrae L1). At the time of extraction, there is also a case where each bone is extracted as a mask region in a connected state. The mask region is a region which is the target of each processing. The model setting unit 163 separates the bones at the position of the joint, for example, using a bone separation algorithm to create mask regions that separate the tarsus 11, the tibia 12, the femur 13, the pelvis 14, and the lumbar vertebrae 15, respectively. The model setting unit 163 can generate the shape of each bone in the kinematic model by the contour of the mask region of each bone.

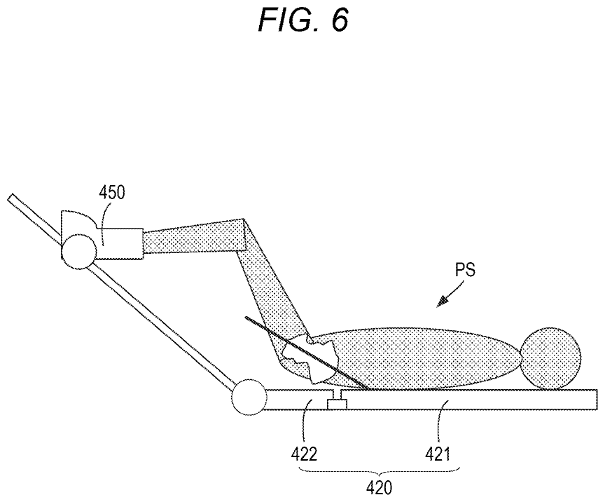

FIG. 5 is a view illustrating an example of a state of the pelvis 14 in a state where the body position of the subject PS is the lithotomy position and the leg part 31 is raised low. FIG. 6 is a view illustrating an example of a state of the pelvis 14 in a state where the body position of the subject PS is the lithotomy position and the leg part 31 is raised high.

In the lithotomy position, the subject is placed facing upward on the surgical bed 400, and on the surgical bed 400, the leg holding unit 450 to which the leg part 31 of the subject PS is fixed is arranged at a higher position than the table 420 on which the body part 33 of the subject PS is placed. The leg of the subject PS is fixed to the leg holding unit 450 in a raised state.

The leg holding unit 450 may have a leg placing unit for placing the leg part 31 on, and a leg fixing unit for fixing the leg part 31 to the leg placing unit. The leg fixing unit may be able to fix the position and orientation of the leg, for example, may fix the position and orientation by straps, or may be worn by inserting the leg part 31 like a boot. In a case of the leg fixing unit such as a boot, the leg placing unit for simply placing the leg part 31 may not be provided.

When the leg part 31 of the subject PS is moved, the pelvis 14 of the subject PS is moved. The pelvic organ 51 moves in conjunction with the pelvis 14. Since the leg part 31 is held by the leg holding unit 450, the position of the leg part 31 substantially matches the position of the leg holding unit 450. The body position and posture of the subject PS can be determined by the relationship between the table 420 and the leg holding unit 450.

The deformation processing unit 162 may calculate the deformation (for example, movement, rotation) of the kinematic model based on the forces (for example, gravity, a force based on the body position) acting on the kinematic model. Accordingly, it is possible to grasp how each bone is deformed based on the body position and posture of the subject PS. The position processing unit 164 may calculate the state (for example, position, orientation (angle)) of the tarsus 11, the tibia 12, the femur 13, the pelvis 14, and the lumbar vertebrae 15 based on the deformation of the kinematic model. For example, the position processing unit 164 may estimate the state (for example, position, orientation, movement) of the pelvis 14 based on the position of the leg holding unit 450 with respect to the table 420 and the kinematic model of the lower limb of the subject PS.

The deformation processing unit 162 may calculate the state (for example, position, orientation (angle)) of each bone (for example, the pelvis 14) of the subject PS when the leg part 31 is moved. In this case, the movement of the kinematic model may be calculated based on the change in the positional relationship between the leg holding unit 450 and the table 420. For example, the model setting unit 163 may additionally generate a model of the leg holding unit 450 and a model of the table 420 in addition to the kinematic model. The deformation processing unit 162 may calculate the movement of the kinematic model using inverse kinematics based on the kinematic model, the positional information on each model (for example, the kinematic model, the model of the leg holding unit 450, and the model of the table 420), and the detection information on the position and angle obtained by the sensor SR of the surgical bed 400. Accordingly, the robotically-assisted surgical device 100 can estimate the state and movement of each bone in the subject PS when the angle of the support member 440 to the second table 422 and the angle of the leg holding unit 450 with respect to the support member 440 are changed. Therefore, the robotically-assisted surgical device 100 can further improve the accuracy of deformation of the pelvic organ 51 in the 3D data by taking into account the movement of the bone such as the pelvis 14 as well as the distance and angle of the leg holding unit 450 with respect to the table 420.

The deformation processing unit 162 may calculate the deformation of the pelvic organ 51 in the 3D data based on the state of the pelvis 14. The deformation here may include movement and rotation. The deformation of the pelvic organ 51 may be derived from the 3D data by large deformation simulation using the finite element method. For example, the deformation processing unit 162 may calculate the force acting on the pelvic organ 51 according to the state of the pelvis 14, and deform the organ 50 based on the force acting on each point of the pelvic organ 51.

Accordingly, the organ deformation in accordance with the body position in TAMIS can be performed based on the volume data obtained by imaging with the CT scanner 200 in the supine position. Accordingly, even when preoperative CT images are used, the state of the pelvic organs 51 corresponding to the body position in TAMIS can be reproduced in the volume data or model, and the surgical accuracy of robotic surgery by TAMIS can be improved.

The image generation unit 166 may generate a rendering image reflecting the deformation of the pelvic organ 51. The display control unit 167 may display the rendering image. Accordingly, the operator can confirm the state of the pelvic organs 51 corresponding to the body position in TAMIS by displaying the state.

In this manner, based on the kinematic model, the robotically-assisted surgical device 100 can calculate, for example, how the bones connected to the joints of the lower limb move according to the bending curvature of the joints of the lower limb. Since the bone is a rigid body, the deformation processing unit 162 may calculate the shape of the bone after the joint movement according to the movement of the joints of the lower limb.

Next, the details of the form of the surgical bed 400 will be described using FIGS. 7A to 7O.

In FIGS. 7A to 7O, similar to FIGS. 5 and 6, it is assumed that the subject PS is placed on the surgical bed 400 in the lithotomy position. In a body position other than the lithotomy position, the form of the surgical bed 400 can be different from that in the examples of FIGS. 7A to 7O. In the lithotomy position, the form of the surgical bed 400 may be different from that of FIGS. 7A to 7O.

In FIGS. 7A to 7O, in order to make it simple to describe, the orientation of the surgical bed 400 is illustrated using the subject coordinate system. In other words, in the surgical bed 400, the x-direction is a direction corresponding to the left-right direction of the subject PS, the y-direction is a direction corresponding to the thickness direction of the subject PS, and the z-direction is a direction corresponding to the body axial direction of the subject PS. The positive side in the y-direction (the ventral side of the subject PS) is described as an upper side, and the negative side in the y-direction (the dorsal side of the subject PS) is described as a lower side. The positive side in the x-direction (the right side of the subject PS) is described as a right side, and the negative side in the y-direction (the left side of the subject PS) is described as a left side. In FIGS. 7D to 7O, there is a case where some parts of the surgical bed 400 are omitted.

FIG. 7A is a side view of the surgical bed 400 viewed from the x-direction. FIG. 7B is a side view of the surgical bed 400 viewed from the z-direction. FIG. 7C is an upper view of the surgical bed 400 viewed from the y-direction.

The base 410 is expandable or contractible, for example, along an expanding/contracting direction m1 which is the vertical direction. Therefore, the height of the table 420, including the first table 421 and the second table 422, is adjustable. The base 410 includes the expansion/contraction mechanism EM for expansion and contraction in the expanding/contracting direction m1, the actuator AC0 that provides a driving force to the expansion/contraction mechanism EM, the sensor SR1 that detects the position of the expansion/contraction mechanism EM, and the like. The position of the expansion/contraction mechanism EM corresponds to the height of the first table 421 in the vertical direction. A rotary joint 461 is connected to the upper end of the base 410.

The first table 421 is connected to the upper end of the rotary joint 461. Prismatic joints 460 are connected to each of the two ends of the rotary joint 461 in the x-direction. The first table 421 and the prismatic joint 460 are rotatable with the directions of three axes as the rotation centers with respect to the rotary joint 461. The directions of three axes may be two perpendicular directions along the horizontal direction and a vertical direction perpendicular to the horizontal direction, and may correspond to the x-direction, the y-direction, and the z-direction with respect to the subject PS. The rotation direction by the rotary joint 461 includes a rotation direction r11 with the x-direction as the rotation center, a rotation direction r12 (FIG. 7B) with the z-direction as the rotation center, and a rotation direction r13 with the y-direction as the rotation center.

The rotary joint 461 includes the rotation mechanism RM1 of which the directions of three axes are the rotation centers, the actuator AC1 that provides a driving force to the rotation mechanism RM1, the sensor SR1 that detects the rotation angle of the rotation mechanism RM1, and the like. The rotation mechanism RM1 may rotate in any directions of two axes or one axis among the directions of three axes as the rotation centers, instead of rotating with the directions of three axes as the rotation centers.

The prismatic joint 460 is connected to the first table 421 at the lower part or the side part of the first table 421. The prismatic joint 460 extends along the z-direction at each of the two end parts of the first table 421 in the x-direction. The first table 421 is movable along a moving direction m2, which is one direction (for example, z-direction) of the first table 421, by the prismatic joint 460.

The prismatic joint 460 includes the slide mechanism SM1 for sliding the first table 421 along the z-direction with respect to the base 410, the actuator AC2 that provides a driving force to the slide mechanism SM1, the sensor SR2 that detects the slide position in the slide mechanism SM1, and the like. The slide position corresponds to the position of the first table 421 in the z-direction with respect to the base 410.

The rotary joint 462 is connected to the end part of the first table 421 on the negative side in the z-direction, and is connected to the end part of the second table 422 on the positive side in the z-direction. The rotary joint 462 rotatably connects the first table 421 and the second table 422 to each other with the y-direction as the rotation center. For example, the second table 422 is rotatably connected to the first table 421. The rotary joint 462 includes the rotation mechanism RM2 of which the y-direction is the rotation center, the actuator AC3 that provides a driving force to the rotation mechanism RM2, the sensor SR3 that detects the rotation angle of the rotation mechanism RM2, and the like. In other words, the second table 422 is rotatable in a rotational direction r4 following the rotation of the rotation mechanism RM2. Accordingly, the leg of the subject can be abducted. In other words, the leg holding unit 450 may have a mechanism that abducts and holds the leg part 31 of the subject PS.

The rotary joint 463 is connected to the end part on the negative side in the z-direction and the end part on the positive side in the x-direction in the second table 422, and to the end part on the positive side in the z-direction in the support member 440. The rotary joint 463 rotatably connects the second table 422 and the support member 440 to each other with the x-direction as the rotation center. For example, the support member 440 is rotatably connected to the second table 422. The rotary joint 463 includes the rotation mechanism RM3 of which the x-direction is the rotation center, the actuator AC4 that provides a driving force to the rotation mechanism RM3, the sensor SR4 that detects the rotation angle of the rotation mechanism RM3, and the like. In other words, the rotary joint 463 is rotatable in a rotational direction r2 following the rotation of the rotation mechanism RM3.

The support member 440 adjustably supports the position of the leg holding unit 450 with respect to the second table 422. The support member 440 has a prismatic joint 464 for adjusting the distance between the second table 422 and the leg holding unit 450 along the extending direction of the support member 440. The prismatic joint 464 may be provided separately from the support member 440 and disposed along the support member 440 in the vicinity of the support member 440.

The prismatic joint 464 can move the leg holding unit 450 along a moving direction m3 along the support member 440. The prismatic joint 464 includes the slide mechanism SM2 for sliding the leg holding unit 450 along the moving direction m3 with respect to the second table 422, the actuator AC5 that provides a driving force to the slide mechanism SM2, the sensor SR5 that detects the slide position in the slide mechanism SM2, and the like. This slide position corresponds to the position of the leg holding unit 450 with respect to the second table 422 along the moving direction m3 and corresponds to the position of a rotary joint 465 to which the leg holding unit 450 is connected.

The rotary joint 465 may be connected to the slide position in the slide mechanism SM2 of the prismatic joint 464 and may be connected to the end part of the leg holding unit 450. The rotary joint 465 rotatably connects the support member 440 to the leg holding unit 450 with the x-direction as the rotation center. For example, the leg holding unit 450 is rotatably connected to the support member 440. The rotary joint 465 includes the rotation mechanism RM4 of which the x-direction is the rotation center, the actuator AC6 that provides a driving force to the rotation mechanism RM4, the sensor SR6 that detects the rotation angle of the rotation mechanism RM4, and the like. In other words, the rotary joint 465 is rotatable in a rotational direction r3 following the rotation of the rotation mechanism RM4. The rotary joint 465 may be left in a free state. The free state here may be defined as a state where the leg holding unit 450 is rotatable, but the rotary joint 465 does not have the actuator AC6 and the rotation angle of the rotation mechanism RM4 is not particularly fixed. In this case, the sensor SR6 may or may not detect the rotation angle of the rotary joint 465. This is because the constraints of the links in the kinematic model make it possible to infer the rotation angle of the rotary joint 465 by information from other sensors.

Since it is assumed that the subject PS has a pair of left and right leg parts 31 and thigh parts 32, as illustrated in FIG. 7C, the surgical bed 400 has two (a pair) parts for left and right for placing the lower limbs. The parts for placing the lower limbs may include, for example, the rotary joint 462, the second table 422, the rotary joint 463, the support member 440, the prismatic joint 464, the rotary joint 465, and the leg holding unit 450.

FIGS. 7D to 7O illustrate a state where the form of the surgical bed 400 is changed from the state illustrated in FIGS. 7A to 7C by each mechanism provided in the surgical bed 400. Here, some of the parts of the surgical bed 400 can be omitted.

FIG. 7D illustrates a state where the base 410 is expanded by the expansion of the expansion/contraction mechanism EM. FIG. 7E is a side view of the table 420 in a state of being slid to the negative side in the z-direction (leg part side of the subject PS) by the slide of the slide mechanism SM1, viewed from the x-direction. FIG. 7F is a side view of the table 420 in a state of being slid to the positive side in the z-direction (head part side of the subject PS) by the slide of the slide mechanism SM1, viewed from the x-direction.

FIG. 7G is a side view of the table 420 in a state of being tilted with the positive side in the z-direction (head part side of the subject PS) lowered by the rotation of the rotation mechanism RM1, viewed from the x-direction. FIG. 7H is a side view of the table 420 in a state of being tilted with the positive side in the z-direction raised by the rotation of the rotation mechanism RM1, viewed from the x-direction. FIG. 7I is a side view of the table 420 in a state of being tilted with the positive side in the x-direction (right side of the subject PS) lowered by the rotation of the rotation mechanism RM1, viewed from the z-direction. FIG. 7J is a side view of the table 420 in a state of being tilted with the positive side in the x-direction raised by the rotation of the rotation mechanism RM1, viewed from the z-direction.

FIG. 7K is a top view of the surgical bed in a leg-opened state by the rotation of the rotation mechanism RM1, viewed from the y-direction. The leg-opened state of the surgical bed 400 is a state where each of the left and right parts for placing the lower limbs of the subject PS is placed away from each other so as to match the leg-opened state of the subject PS. FIG. 7L is a top view of the surgical bed 400 in a leg-closed state, viewed from the y-direction. The leg-closed state of the surgical bed 400 is a state where each of the left and right parts for placing the lower limbs of the subject PS is placed close to each other so as to match the leg-closed state (a state where the legs are not opened) of the subject PS.

FIG. 7M is a top view of a state where the distance between the table 420 and the leg holding unit 450 is increased by the slide of the slide mechanism SM2, viewed from the x-direction. FIG. 7N is a side view of a state where the angle of the support member 440 with respect to the horizontal direction is increased and the support member 440 is largely tilted with respect to the horizontal direction, viewed from the x-direction. FIG. 7O is a top view of a state where the angle of the leg holding unit 450 is adjusted by the rotation of the rotation mechanism RM4, viewed from the x-direction. In FIG. 7O, the leg holding unit 450 has been adjusted to follow the horizontal direction from the state in FIG. 7N.

In FIGS. 7A to 7O, the form of the surgical bed 400 that assumes the lithotomy position is assumed, but the subject PS in other body positions can also be placed and fixed on the surgical bed 400. For example, in the surgical bed 400, the subject PS in the supine position with legs abducted, prone position, Trendelenburg position, reverse Trendelenburg position, jackknife position, and other body positions can be placed and fixed. The subject PS will not be in a body position of sitting on the surgical bed 400, that is, the surgical bed 400 will not be in a chair-like form. This is because the technique for approaching the pelvic organ 51 from the groin is mainly assumed.

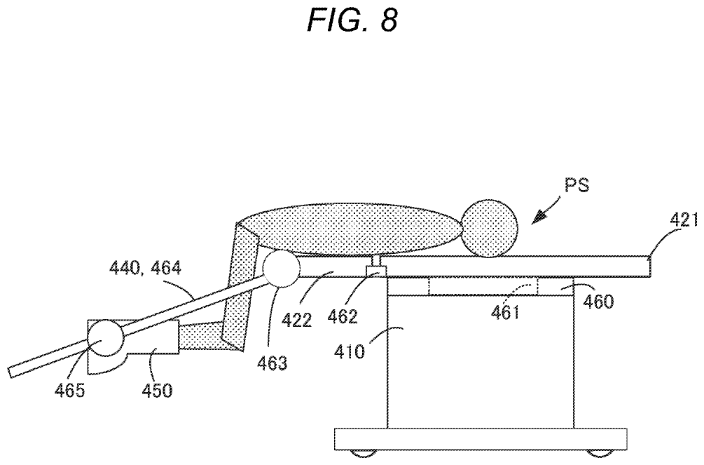

In the jackknife position, the subject is placed in a prone position on the surgical bed 400, and on the surgical bed 400, the leg holding unit 450 is arranged at a lower position than the table 420. The leg part 31 of the subject PS is fixed to the leg holding unit 450 in a lowered state. FIG. 8 is a view illustrating an example of a form of the surgical bed 400 corresponding to a jackknife position. This form can be realized by adjusting the rotation amount of the rotation mechanism RM3 of the rotary joint 463 and the rotation mechanism RM4 of the rotary joint 465. Furthermore, on the surgical bed 400, a placing table for placing the shin part 31b of the subject PS thereon may be provided, or a part of the support member 440 may be configured to make it possible to place the shin part 31b thereon. In this case, the position of the shin part 31b becomes even more stable, and the body position of the subject PS becomes even more stable.

In a case where the body position of the subject PS is in a lithotomy position or jackknife position on the surgical bed 400, the second table 442 is provided and the second table 442 may not be used. In this case, the rotary joint 463 with the x-direction as the rotation center may be connected to the rotary joint 462 with the y-direction as the rotation center. The rotary joint 462 and the rotary joint 463 may be integrated to make it possible to rotate with two directions (the x-direction and the y-direction) as the rotation center with one rotation mechanism.

Each unit (for example, the actuator AC, the sensors SR) provided in each prismatic joint and each rotary joint may be provided in locations other than the joints. At least two actuators AC or sensors SR which are provided in each prismatic joint and each rotary joint may be shared.

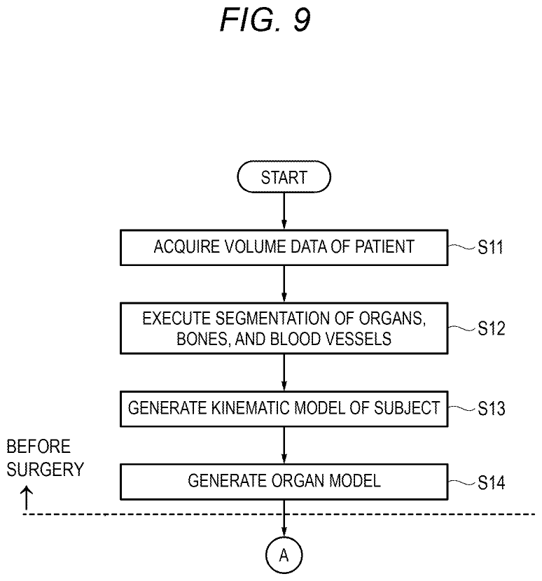

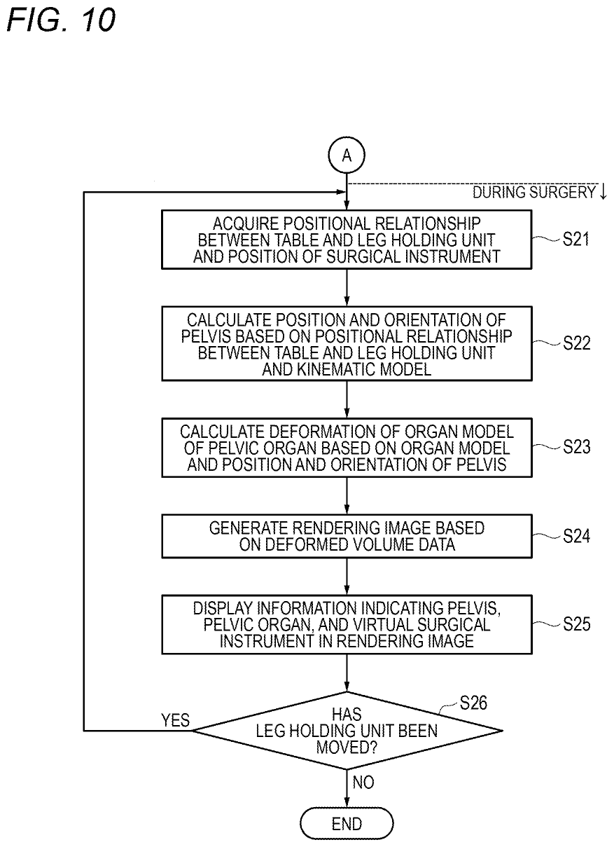

FIGS. 9 and 10 are flowcharts illustrating an operation example of the robotically-assisted surgical device 100. S11 to S14 in FIG. 9 are executed, for example, before surgery, and S21 to S26 in FIG. 10 are executed, for example, during surgery. Each processing here is executed, for example, by each unit of the processing unit 160.

First, before surgery, the volume data of the subject PS (for example, a patient) is acquired (S11). Segmentation to extract regions of organs, bones, and blood vessels is executed (S12). Based on the volume data, the kinematic model of the subject PS (for example, a kinematic model of the lower limb) is generated. Based on the volume data, the organ model of the pelvic organ 51 of the subject PS is generated (S14).

When the robotic surgery is started, the surgical bed 400 on which the surgical robot 300 and the subject PS are placed is arranged at a predetermined position. During surgery, the surgical instrument 30 is inserted into the subject PS via the platform 40 installed on the anus.

Then, the information on the positional relationship between the table 420 and the leg holding unit 450 is acquired, for example, via the transmission/reception unit 110, and the position of the surgical instrument 30 is acquired (S21). The position and orientation of the pelvis 14 are calculated based on the positional relationship between the table 420 and the leg holding unit 450, and the kinematic model (S22). Based on the position and orientation of the pelvis, the deformation of the organ model corresponding to the pelvic organ 51 is calculated (S23).

The region of the pelvic organ 51 in the volume data deforms corresponding to the deformation of the organ model. For example, the deformation of the pelvic organ 51 corresponding to the elevation status of the leg part 31 of the subject PS is reflected in the volume data. The deformed volume data is rendered to generate a rendering image (for example, virtual endoscopic image) (S24). A first display image is generated by superimposing information illustrating the pelvis 14, the pelvic organ 51, and a virtual surgical instrument 30V on the rendering image. In the virtual endoscopic image, the reflected virtual surgical instrument 30V is a virtual end effector. The generated first display image is displayed on the display 130 or the image display terminal 330 (S25). The actual endoscopic image may be acquired from the endoscope ES via the transmission/reception unit 110, and displayed on the display 130 or the image display terminal 330 as a second display image. The surgical instrument 30 is reflected on the actual endoscopic image.

It is determined whether or not the leg holding unit 450 has been moved in the surgical bed 400 (S26). In this case, it may be determined whether or not the positional relationship between the table 420 and the leg holding unit 450, acquired via the transmission/reception unit 110, has changed. In a case where this positional relationship does not change, it is possible to determine that the body position of the subject PS has not changed, and thus, the processing in FIG. 10 is suspended temporarily. In a case where this positional relationship has changed, it is possible to determine that the body position of the subject PS has changed, and thus, the state of the pelvis 14 or the pelvic organ 51 is supposed to be derived again, and the processing proceeds to S21. When the surgery is completed, the intraoperative processing in FIG. 10 is completed. The completion of the surgery may be instructed to the operator, for example, via the UI 120. When the surgery is completed, for example, the surgical instrument 30 and the platform 40 are removed from the subject PS, the surgical robot 300 is detached from the surgical bed 400, the tubes for anesthesia or blood transfusion are removed from the subject PS, and the wound is sutured after the platform 40 is removed.

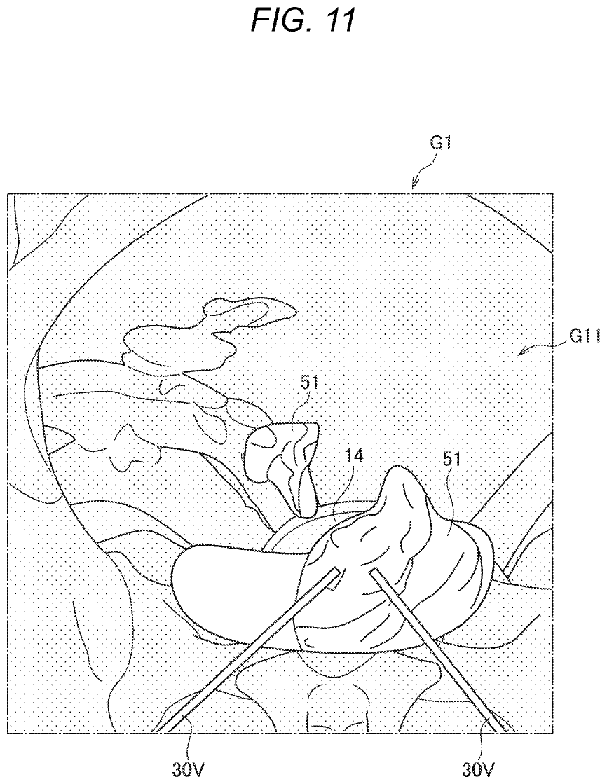

FIG. 11 is a view illustrating an example of a first display image G1. The first display image G1 includes a virtual endoscopic image G11. The virtual endoscopic image G11 illustrates a state of the pelvic organ 51 near the pelvis 14. The first display image G1 illustrates information indicating the pelvis 14, the pelvic organ 51, and the virtual surgical instrument 30V together with the virtual endoscopic image G11. In the first display image G1, the virtual surgical instrument 30V is illustrated at the image position in the virtual endoscopic image G11 corresponding to the position of the surgical instrument 30 in the actual space.

In this manner, since the surgical bed 400 can derive the positional relationship between the table 420 and the leg holding unit 450, the surgical bed 400 and the robotically-assisted surgical device 100 can grasp the elevation status of the leg part 31 of the subject PS corresponding to this positional relationship, or the body position or posture of the subject PS during surgery. The robotically-assisted surgical device 100 can estimate the state of the pelvis 14 in the 3D data based on the body position and posture of the subject PS, and can estimate the deformation of the pelvic organ 51 based on the state of the pelvis 14. In TAMIS, the movement of the pelvis 14 in the subject PS is large and the deformation of the pelvic organ 51 is large as compared with the body position change in other laparoscopic surgeries. In other words, the movement of the pelvis 14 will be greater when changing the body position by raising and lowering the leg holding unit 450 corresponding to the leg part 31 than when changing the body position on the table 420. Even when the pelvic organ 51 moves due to changes in special body position, the deformation (movement) of the organ can be calculated virtually in the 3D data. The robotically-assisted surgical device 100 can then reflect the deformation of the pelvic organ 51 caused by changes in the body position and posture of the subject PS in the volume data. Accordingly, even when the body position of the subject PS corresponding to the volume data obtained before surgery and the body position of the subject PS during surgery differ from each other, the relations between both body positions can be associated with each other, and it is possible to suppress decrease in the surgical accuracy of robotic surgery.

By providing the actuator AC for driving each rotation mechanism RM and each slide mechanism SM, the form of the surgical bed 400 can be automatically changed. Accordingly, for example, the position of the leg holding unit 450 with respect to the table 420 can be easily changed. Therefore, each time the position of the leg holding unit 450 is changed, it is not necessary to remove the leg part 31 of the subject PS from the leg holding unit 450 before the change, and to manually attach the leg part 31 to the leg holding unit 450 after the change. Since the body shapes of subjects PS, such as height, differ from one subject to another, there are many cases where the elevation status of the leg part 31 differs from one subject to another even when the same surgical procedure is used. Even in this case, the robotically-assisted surgical device 100 can reflect the exact body position of the subject PS in the surgical navigation by deriving the positional relationship between the table 420 and the leg holding unit 450, and it is possible to suppress decrease in the surgical accuracy of robotic surgery.

Even when the body position is changed in a state (docked state) where the surgical instrument 30 is inserted into the body of the subject PS during surgery by recognizing the movement of the pelvis 14 or the pelvic organ 51, the robotically-assisted surgical device 100 can estimate the movement of the pelvis 14 and the pelvic organ 51 of the subject PS that cannot be visually recognized from the outside, and can continue the robotic surgery. The operator can also grasp the movement of the pelvis 14 to make it easier to perform muscle detachment near the anus, for example.

Although various embodiments have been described above with reference to the drawings, it is needless to say that the present disclosure is not limited to such examples. It is clear that a person skilled in the art can come up with various changes or modifications within the scope of the claims, and it is understood that these changes or modifications naturally belong to the technical scope of the present disclosure.

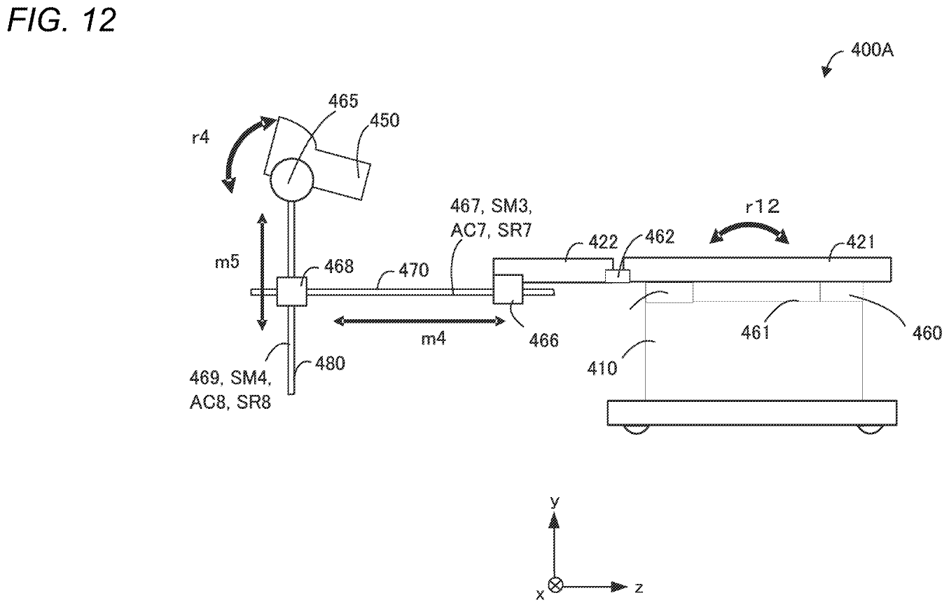

The aspect of the surgical bed 400 is not limited to the aspect illustrated in FIGS. 7A to 7O. For example, the aspect illustrated in FIG. 12 may be employed. FIG. 12 is a view illustrating a deformation configuration example (a surgical bed 400A) of the surgical bed 400. In FIG. 12, the description of the same items as in FIGS. 7A to 7O will be omitted or simplified.

When comparing to the surgical bed 400 of FIG. 7A and the like, the surgical bed 400A of FIG. 12 does not have the rotary joint 463, has connection units 466 and 468, and has support members 470 and 480 instead of the support member 440.

The connection unit 466 is connected to the end part on the negative side in the z-direction and the end part (lower end part) on the negative side in the y-direction of the second table 422, and is connected to the positive side of the support member 470 in the z-direction. The connection unit 466 connects the second table 422 and the support member 470 to each other.

The connection unit 468 is connected to any position in the support member 470 and to any position in the support member 480. The connection unit 468 connects the support member 470 and the support member 480 to each other.

The support member 470 adjustably supports the position of the connection unit 468 with respect to the second table 422. The support member 440 has a prismatic joint 467 for adjusting the distance of the connection unit 468 from the second table 422 along the extending direction of the support member 440. The prismatic joint 467 may be provided separately from the support member 470 and disposed along the support member 470 in the vicinity of the support member 470. The extending direction of the support member 440 is a direction along the z-direction in the leg-closed state of the surgical bed 400A.

The prismatic joint 467 can move the connection unit 468 along a moving direction m4 along the support member 470. The prismatic joint 467 includes the slide mechanism SM3 for sliding the connection unit 468 along the moving direction m3 with respect to the second table 422, the actuator AC7 that provides a driving force to the slide mechanism SM3, the sensor SR7 that detects the slide position in the slide mechanism SM3, and the like. This slide position corresponds to the position of the connection unit 468 with respect to the second table 422 along the moving direction m4.

The support member 480 adjustably supports the position of the connection unit 468 with respect to the leg holding unit 450. The rotary joint 465 connected to the leg holding unit 450 is connected and fixed to the end part of the support member 480 on the positive side in the y-direction. The support member 480 has a prismatic joint 469 for adjusting the distance between the leg holding unit 450 and the connection unit 468 along the extending direction of the support member 480. The prismatic joint 469 may be provided separately from the support member 480 and disposed along the support member 480 in the vicinity of the support member 480. The extending direction of the support member 480 is along the y-direction.

The prismatic joint 469 can move the connection unit 468 along a moving direction m5 along the support member 480. The prismatic joint 469 includes the slide mechanism SM4 for sliding the connection unit 468 along the moving direction m5 with respect to the leg holding unit 450, the actuator AC8 that provides a driving force to the slide mechanism SM4, the sensor SR8 that detects the slide position in the slide mechanism SM4, and the like. This slide position corresponds to the position of the connection unit 468 with respect to the leg holding unit 450 along the moving direction m5.

Therefore, the slide position in the slide mechanism SM3 and the slide position in the slide mechanism SM4 determine the positional relationship between the second table 422 and the leg holding unit 450. Accordingly, the processor PR can derive the positional relationship between the second table 422 and the leg holding unit 450 based on the detection results by the sensors SR6, SR7, and SR8.

In this manner, instead of adjusting the angle of the support member 440 with respect to the second table 422, the surgical bed 400A may be provided with two support members 470 and 480, and the connection unit 468 that connects the support members 470 and 480 to each other may be movable along the support members 470 and 480. Accordingly, the surgical bed 400A can adjust the distance between the second table 422 and the leg holding unit 450 to a desired distance, and adjust the angle of the leg holding unit 450 with respect to the second table 422 to a desired angle.

An example is illustrated in which the processor PR of the surgical bed 400 derives the positional relationship between the table 420 and the leg holding unit 450 based on the measurement results from the linear encoder or the rotary encoder, but the disclosure is not limited thereto. For example, the sensor SR may measure the three-dimensional position of the leg holding unit 450 and the three-dimensional position of the table 420. The processor PR may acquire the measured three-dimensional position. The processor PR may calculate the position of the leg holding unit 450 with respect to the table 420 based on the three-dimensional position of the leg holding unit 450 and the three-dimensional position of the table 420.

The processor PR may derive the positional relationship between the table 420 and the leg holding unit 450 using optical methods. For example, an optical marker MK1 may be attached to any position (for example, near the rotary joint 465 in the leg holding unit 450) in the leg holding unit 450, and an optical marker MK2 may be attached to any position (for example, near the rotary joint 463 in the second table 422) in the table 420. An imaging device may be installed at any position in the operating room where the surgical bed 400 is placed. Any position mentioned here may include a wall surface or ceiling of the operating room, a position suspended from the ceiling, any surface of the surgical bed 400, a side surface of the surgical robot 300, a side surface of various carts used in the robotic surgery, or the like. The optical markers MK1 and MK2 are positioned in the imaging range of the imaging device. The optical markers MK1 and MK2 emit light when being irradiated with infrared light from the imaging device or the like. As a result, the optical markers MK1 and MK2 are reflected in the captured image of the imaging device.

The processor PR may derive the positional relationship between the table 420 and the leg holding unit 450 using a three-dimensional position sensor. For example, a magnetic probe MK11 may be attached to any position (for example, near the rotary joint 465 in the leg holding unit 450) in the leg holding unit 450, and a magnetic probe MK12 may be attached to any position (for example, near the rotary joint 463 in the second table 422) in the table 420. A magnetic three-dimensional position sensor may be installed at any position in the operating room where the surgical bed 400 is placed. Any position mentioned here may include a wall surface or ceiling of the operating room, a position suspended from the ceiling, any surface of the surgical bed 400, a side surface of the surgical robot 300, a side surface of various carts used in the robotic surgery, or the like. When the magnetic probes MK11 and MK12 are positioned in the measurement range of the magnetic three-dimensional position sensor, the three-dimensional position sensor acquires the coordinates of the magnetic probes MK11 and MK12.

The processor PR may derive the positional relationship between the table 420 and the leg holding unit 450 using an acceleration sensor and a gyro. For example, an acceleration sensor and a gyro MK21 may be attached to any position (for example, near the rotary joint 465 in the leg holding unit 450) in the leg holding unit 450, and an acceleration sensor and a gyro MK22 may be attached to any position (for example, near the rotary joint 463 in the second table 422) in the table 420. The acceleration sensors and the gyros MK21 and MK22 are initialized to coordinate at a predetermined origin with respect to the second table 422. After this, when the acceleration sensors and the gyros MK21 and MK22 move, the acceleration sensors and the gyros MK21 and MK22 wirelessly transmit the relative coordinates with respect to the second table 422 to the robotically-assisted surgical device 100.

The processor PR acquires, via the communication unit 405, the image captured by the imaging device and the additional information of the captured image. The additional information may include information regarding the imaging (for example, imaging position, imaging orientation, viewing angle, imaging range, and imaging time). The processor PR recognizes the position (image position) of the leg holding unit 450 in the captured image and the position (image position) of the table 420 in the captured image, based on the positions of the optical markers MK1 and MK2 reflected in the captured image. The processor PR can recognize the position of the leg holding unit 450 and the position of the table 420 in real space based on the image position of the leg holding unit 450 and the image position of the table 420 in the captured image. The recognition of this position corresponds to the measurement of the position of the leg holding unit 450 and the position of the table 420. Accordingly, the processor PR can derive the positional relationship between the leg holding unit 450 and the table 420.

In a case where the sensor SR and the imaging device that captures the optical marker are provided in the surgical bed 400, calibration in the operating room is not necessary. In other words, since the surgical robot 300 is connected to the surgical bed 400, the positional relationship between the surgical robot 300 and the surgical bed 400 is grasped in the robotically-assisted surgical system 1. Accordingly, the position processing unit 164 can align the coordinate system of the surgical robot 300 including the surgical bed 400 with the coordinate system of the subject PS using the sensor SR or the imaging device of the surgical bed 400, and calibration of both coordinate systems becomes unnecessary.

In a case where the imaging device is installed on the table 420, the optical marker MK2 attached to the table 420 is not necessary. Even in this case, the position of the leg holding unit 450 with respect to the table 420 can be recognized based on the captured image. In other words, the processor PR can derive the positional relationship between the table 420 and the leg holding unit 450 by recognizing the image position of the leg holding unit 450 reflected in the captured image.

Instead of the surgical bed 400, the robotically-assisted surgical device 100 may derive the positional relationship between the table 420 and the leg holding unit 450 using optical methods. In this case, the processor 140 may operate instead of the processor PR, and the transmission/reception unit 110 may operate instead of the communication unit 405.

Although the above-described embodiments can be applied to TAMIS, the embodiment may be applied to other surgery procedures, for example, to Transanal Total Mesenteric Excision (TaTME). The above-described embodiment can be applied to the rectum, but may be applied to surgical procedures for the prostate, uterus, bladder, other pelvic organs, surrounding organs, tissues, or joints.

The embodiments can be used not only for the robotic surgery based on the operation of the operator, but also for autonomous robotic surgery (ARS) or semi-ARS. ARS is a fully automatic robotic surgery performed by an AI-equipped surgical robot. Semi-ARS basically automatically performs the robotic surgery by an AI-equipped surgical robot, and partially performs the robotic surgery by the operator.

Although the endoscopic surgery by the robotic surgery is exemplified, the surgery may be performed by the operator directly operating the surgical instrument 30. In this case, the robot main body 320 may be the subject PS, the robot arm AR may be the arm of the operator, and the surgical instrument 30 may be forceps and an endoscope that the operator grips and uses for treatment.

The preoperative simulation and the intraoperative navigation may be configured by a separate robotically-assisted surgical device. For example, the preoperative simulation may be performed by a simulator, and the intraoperative navigation may be performed by a navigator.

The robotically-assisted surgical device 100 may include at least the processor 140 and the memory 150. The transmission/reception unit 110, the UI 120, and the display 130 may be externally attached to the robotically-assisted surgical device 100.

It is exemplified that the volume data as the captured CT image is transmitted from the CT scanner 200 to the robotically-assisted surgical device 100. Instead of this, the volume data may be transmitted to and stored in a server (for example, an image data server (PACS) (not illustrated)) or the like on the network such that the volume data is temporarily stored. In this case, the transmission/reception unit 110 of the robotically-assisted surgical device 100 may acquire the volume data from a server or the like via a wired circuit or a wireless circuit when necessary, or may acquire the volume data via any storage medium (not illustrated).

It is exemplified that the volume data as the captured CT image is transmitted from the CT scanner 200 to the robotically-assisted surgical device 100 via the transmission/reception unit 110. This also includes a case where the CT scanner 200 and the robotically-assisted surgical device 100 are established by being substantially combined into one product. This also includes a case where the robotically-assisted surgical device 100 is handled as the console of the CT scanner 200. The robotically-assisted surgical device 100 may be provided in the surgical robot 300. The robotically-assisted surgical device 100 may be provided in the surgical bed 400.

Although it is exemplified that the CT scanner 200 is used to capture an image and the volume data including information on the inside of the subject is generated, the image may be captured by another device to generate the volume data. Other devices include a magnetic resonance imaging (MM) device, a positron emission tomography (PET) device, a blood vessel imaging device (angiography device), or other modality devices. The PET device may be used in combination with other modality devices.

A robotically-assisted surgical method in which the operation in the robotically-assisted surgical device 100 is defined can be visualized. A program for causing a computer to execute each step of the robotically-assisted surgical method can be visualized.

(Overview of Above-Described Embodiments)

One aspect of the above embodiments is the surgical bed 400 including the table 420, the leg holding unit 450, the support member 440 (an example of a support member), and the processor PR (an example of a processing unit). The body part 33 of the subject PS may be placed on the table 420. The leg holding unit 450 holds the leg part 31 of the subject PS. The support member 440 is connected to the table 420 and the leg holding unit 450, and adjustably supports the positional relationship between the leg holding unit 450 and the table 420. The processor PR has a function of deriving the positional relationship between the leg holding unit 450 and the table 420.

Accordingly, since it is possible to derive the positional relationship between the table 420 and the leg holding unit 450, the surgical bed 400 can grasp the elevation status of the leg part 31 of the subject PS corresponding to this positional relationship, or the body position or posture of the subject PS during surgery. Accordingly, even when the body position of the subject corresponding to the volume data obtained before surgery and the body position of the subject during surgery differ from each other, the surgical bed 400 can improve the surgical accuracy of robotic surgery. Since the position of the leg part 31 held by the leg holding unit 450 is determined by confirming this positional relationship, the surgical bed 400 can reduce the placement error for each placing person who places the subject PS on the surgical bed 400 in a desired body position.

The surgical bed 400 may also include the sensor SR. The support member 440 may adjustably support the angle of the leg holding unit 450 with respect to the table 420 and the distance between the table 420 and the leg holding unit 450. The sensor SR may detect the angle of the leg holding unit 450 with respect to the table 420 and the distance between the table 420 and the leg holding unit 450. The processor PR may derive the positional relationship between the leg holding unit 450 and the table 420 based on the above-described angle and distance. Accordingly, the surgical bed 400 can easily recognize the positional relationship between the leg holding unit 450 and the table 420 by collecting the detection results of one or more sensors SR.

The surgical bed 400 may also include the actuator AC. The leg holding unit 450 may be movable along the support member 440 according to the driving force from the actuator AC. Accordingly, the surgical bed 400 can facilitate the change of the body position including the movement of the leg part 31 that matches the desired body position for the robotic surgery.

The surgical bed 400 may also include the acquisition unit that acquires at least one of information on a planned body position, information on a surgical procedure, and positional information on a surgical instrument. The leg holding unit 450 may be movable according to the control by the actuator AC and based on at least one of the information on the planned body position, the information on the surgical procedure, and the positional information on the surgical instrument 30. Accordingly, the surgical bed 400 can easily set the position of the leg holding unit 450 and adjust the body position or posture that makes it easy to perform robotic surgery on subject PS, according to various pieces of information related to robotic surgery.

The processor PR may derive the positional relationship between the leg holding unit 450 and the table 420 based on the movement of the leg holding unit 450 along the support member 440. Accordingly, the surgical bed 400 can efficiently derive the positional relationship only in a case where, for example, there is a possibility that the leg holding unit 450 moves and the body position or posture of the subject PS is changed, which leads to energy conservation.

One aspect of the above-described embodiment is the robotically-assisted surgical device 100 that assists the endoscopic surgery by the surgical robot 300. The processing unit 160 of the robotically-assisted surgical device 100 has a function of acquiring information on the positional relationship between the leg holding unit 450 and the table 420 derived by the surgical bed 400, acquiring the 3D data of the subject PS, and estimating at least the state of the pelvis 14 of the subject PS in the 3D data based on the positional relationship between the leg holding unit 450 and the table 420 and the 3D data.

Accordingly, even when the internal state of the subject PS changes due to the change in the body position, for example, the robotically-assisted surgical device 100 can recognize the positional relationship between the leg holding unit 450 and the table 420 and virtually estimate the pelvic state in the 3D data. The robotically-assisted surgical device 100 can reflect the change in the state of the pelvis caused by changes in the body position or posture of the subject PS in the 3D data. Accordingly, even when the body position of the subject PS corresponding to the volume data obtained before surgery and the body position of the subject PS during surgery differ from each other, it is possible to grasp the change in the pelvic state where the position of the organ which is the surgery target can be estimated, and to suppress decrease in the surgical accuracy of robotic surgery.

The processing unit 160 may generate the kinematic model of the lower limb of the subject PS based on the 3D data. The processing unit 160 may estimate the state of the pelvis 14 based on the positional relationship between the leg holding unit 450 and the table 420 and the kinematic model. Accordingly, the robotically-assisted surgical device 100 can improve the estimated accuracy because the kinematic model estimates the state of the pelvis 14 taking into account the positional relationship or linkage of each bone of the lower limb.

The processing unit 160 may calculate the deformation of organ (for example, the pelvic organ 51) which is in conjunction with the pelvis 14 of the subject PS in the 3D data based on the 3D data and the state of the pelvis 14. Accordingly, the robotically-assisted surgical device 100 can calculate the deformation (for example, movement) of organ in conjunction with the state of the pelvis 14. Therefore, it is possible to grasp the state of the pelvic organ 51 in the subject PS with high accuracy even when the body position is changed, and to improve the accuracy of treatment for the pelvic organ 51.

The processing unit 160 may acquire the position of the surgical instrument 30 from the surgical robot 300. The processing unit 160 may generate an image of the subject PS based on the 3D data, display information indicating the pelvis 14 at a position corresponding to the position of the pelvis 14 in the image of the subject PS, and display information indicating the surgical instrument 30 at a position corresponding to the position of the surgical instrument 30 in the image of the subject PS. Accordingly, the robotically-assisted surgical device 100 can display information indicating the state of the pelvis 14, the state of deformed organ, and the surgical instrument 30, together with image of the subject PS. Therefore, by confirming the display, the operator can operate the surgical instrument 30 in accordance with the change in body position caused by the movement of the leg holding unit 450, for example, to perform the robotic surgery with high accuracy.

According to another aspect of the above-described embodiment, there is provided a robotically-assisted surgical method that assists endoscopic surgery by the surgical robot 300, including: a step of acquiring a positional relationship between the table 420 and the leg holding unit 450 that are provided in the surgical bed 400 on which the subject PS is placed; a step of acquiring 3D data of the subject PS; and a step of estimating at least a state of the pelvis 14 of the subject PS in the 3D data based on the positional relationship between the leg holding unit 450 and the table 420 and the 3D data.

According to still another aspect of the embodiment, there is provided a program for causing a computer to execute the above-described robotically-assisted surgical method.

The present disclosure is advantageous in the surgical bed, the robotically-assisted surgical device, the robotically-assisted surgical method, and the system that can suppress a decrease in the surgical accuracy of robotic surgery even when the body position of the subject corresponding to the volume data obtained before surgery and the body position of the subject during surgery differ from each other.

1. A surgical bed comprising:
a table on which a body part of a subject is placed;
a leg holder configured to hold a leg part of the subject;
a support member configured to connect the table and the leg holder to each other, and configured to adjustably support a positional relationship between the leg holder and the table; and
a processor configured to derive the positional relationship between the leg holder and the table.

2. The surgical bed according to claim 1, further comprising:
a sensor, wherein
the support member is configured to adjustably support an angle of the leg holder with respect to the table and a distance between the table and the leg holder,
the sensor is configured to detect the angle of the leg holder with respect to the table and the distance between the table and the leg holder, and
the processor is configured to derive the positional relationship between the leg holder and the table based on the angle and the distance.

3. The surgical bed according to claim 1, further comprising:
an actuator, wherein
the leg holder is movable along the support member according to a driving force from the actuator.

4. The surgical bed according to claim 3, further comprising:
an acquisition unit that acquires at least one of information on a planned body position, information on a surgical procedure, and positional information on a surgical instrument, wherein
the leg holder is movable based on control by the actuator and based on at least one of the information on the planned body position, the information on the surgical procedure, and the positional information on the surgical instrument.

5. The surgical bed according to claim 1, wherein
the processor is configured to derive the positional relationship between the leg holder and the table based on movement of the leg holder along the support member.

6. The surgical bed according to claim 1, wherein
the leg holder is configured to fix the leg part of the subject in a lithotomy position.

7. The surgical bed according to claim 1, wherein
the leg holder has a mechanism to abduct and hold the leg part of the subject.

8. A robotically-assisted surgical device that assists endoscopic surgery by a surgical robot, comprising:
a processor, wherein
the processor is configured to:
acquire information on a positional relationship between the leg holder and the table derived by the surgical bed according to claim 1,
acquire 3D data of a subject, and
estimate at least a state of pelvis of the subject in the 3D data based on the positional relationship between the leg holder and the table and the 3D data.

9. The robotically-assisted surgical device according to claim 8, wherein
the processor is configured to calculate deformation of organs which are in conjunction with the pelvis of the subject in the 3D data based on the 3D data and the state of the pelvis.

10. The robotically-assisted surgical device according to claim 8, wherein
the processor is configured to:
acquire a position of a surgical instrument from the surgical robot;
generate an image of the subject based on the 3D data; and
display information indicating the pelvis at a position corresponding to a position of the pelvis in the image of the subject, and display information indicating the surgical instrument at a position corresponding to the position of the surgical instrument in the image of the subject.

11. A robotically-assisted surgical method for assisting endoscopic surgery by a surgical robot, the robotically-assisted surgical method comprising:
acquiring a positional relationship between a table and a leg holder that are provided in a surgical bed on which a subject is placed;
acquiring 3D data of the subject; and
estimating at least a state of pelvis of the subject based on the positional relationship between the leg holder and the table and the 3D data.

12. The method according to claim 11, wherein
the surgical bed comprises a sensor and a support member configured to connect the table and the leg holder to each other,
the support member is configured to adjustably support an angle of the leg holder with respect to the table and a distance between the table and the leg holder, and
the sensor is configured to detect the angle of the leg holder with respect to the table and the distance between the table and the leg holder, and
the positional relationship between the leg holder and the table is acquired based on the angle and the distance.

13. The method according to claim 12, wherein
the surgical bed comprises an actuator, and
the leg holder is movable along the support member according to a driving force from the actuator.

14. A system comprising:
a surgical robot;
a surgical bed;
a computed tomography scanner; and
a robotically-assisted surgical device that assists endoscopic surgery by the surgical robot, wherein the surgical bed comprises:
a table on which a body part of a subject is placed;
a leg holder configured to hold a leg part of the subject;
a support member configured to connect the table and the leg holder to each other, and configured to adjustably support a positional relationship between the leg holder and the table; and
a first processor configured to derive the positional relationship between the leg holder and the table, wherein the robotically-assisted surgical device comprises: a second processor, wherein
the processor is configured to:
acquire information on a positional relationship between the leg holder and the table derived by the first processor,
acquire 3D data of a subject by using the computed tomography scanner, and
estimate at least a state of pelvis of the subject in the 3D data based on the positional relationship between the leg holder and the table and the 3D data.

15. The system according to claim 14, wherein
the surgical bed comprises a sensor,
the support member is configured to adjustably support an angle of the leg holder with respect to the table and a distance between the table and the leg holder,
the sensor is configured to detect the angle of the leg holder with respect to the table and the distance between the table and the leg holder, and
the first processor is configured to derive the positional relationship between the leg holder and the table based on the angle and the distance.

16. The system according to claim 14, wherein
the surgical bed comprises an actuator, and
the leg holder is movable along the support member according to a driving force from the actuator.

17. The system according to claim 16, wherein
the surgical bed comprises an acquisition unit that acquires at least one of information on a planned body position, information on a surgical procedure, and positional information on a surgical instrument, and
the leg holder is movable based on control by the actuator and based on at least one of the information on the planned body position, the information on the surgical procedure, and the positional information on the surgical instrument.

18. The system according to claim 14, wherein
the first processor is configured to derive the positional relationship between the leg holder and the table based on movement of the leg holder along the support member.

19. The system according to claim 14, wherein
the leg holder is configured to fix the leg part of the subject in a lithotomy position.

20. The system according to claim 14, wherein
the leg holder has a mechanism to abduct and hold the leg part of the subject.
